(19) United States
(12) Patent Application Publication
(10) Pub. No.: US 2025/0332278 A1
(43) Pub. Date: Oct. 30, 2025

(54) MULTIVALENT DISPLAY OF APOAI PEPTIDE ON NANOPARTICLES AGAINST ATHEROSCLEROSIS

(71) Applicant: The Regents of the University of California, Oakland, CA (US)

(72) Inventors: Nicole F. Steinmetz, San Diego, CA (US); Matthew D. Shin, San Diego, CA (US)

(21) Appl. No.: 18/869,799

(22) PCT Filed: May 31, 2023
(86) PCT No.: PCT/US2023/024029

§ 371 Date: Nov. 26, 2024

Related U.S. Application Data
(60) Provisional application No. 63/347,967, filed on Jun. 1, 2022.

(51) Int. Cl. A61K 47/69; A61K 47/60; A61P 9/10; A61P 17/02; C12N 7/00

(52) U.S. Cl. CPC A61K 47/6901; A61K 47/60; A61P 9/10; A61P 17/02; C12N 7/00; C12N 2770/00023; C12N 2795/18123

(57) ABSTRACT

Methods and compositions for the treatment of atherosclerosis or cardiovascular disease are provided herein. The compositions and methods contain a nanoparticle-peptide conjugate that reduces LDL concentration in a subject, reduces reactive oxygen species in the subject, or increases cellular healing rate.

### CROSS-REFERENCE TO RELATED APPLICATION

This application claims priority under 35 U.S.C. § 119(e) to U.S. Provisional Application No. 63/347,967, filed Jun. 1, 2022, the contents of which are incorporated herein by reference in their entireties.

### STATEMENT OF GOVERNMENT SUPPORT

This invention was made with government support under HL137674 and GM112584 awarded by the National Institutes of Health. The government has certain rights in the invention.

### BACKGROUND

Atherosclerosis is a progressive cardiovascular disease (CVD) in which excess cholesterol accrues within the arterial intima as a fibrofatty plaque. In late-stage CVD, thrombosis and plaque rupture can occur, resulting in myocardial infarction or stroke, which are the two leading causes of death worldwide. Moreover, patients who recovered from thrombotic complication or stroke can suffer long-term consequence; the degree and permanence of heart, brain, or organ damage is directly related to the duration of insufficient oxygen supply.

Thus, a need exists in the art for a safe and effective treatment for CVD and associated diseases. This disclosure satisfies this need and provides related advantages as well.

### SUMMARY OF THE DISCLOSURE

Provided herein are multivalent nanoparticle-peptide conjugates, comprising, or consisting essentially of, or yet further consisting of a biomimetic ApoAI peptide chemically conjugated to an outer surface of a nanoparticle. In one aspect, a plurality of biomimetic ApoAI peptides is conjugated to the nanoparticle, the nanoparticle and/or biomimetic ApoAI peptide that may be the same or different from each other.

In one aspect, the nanoparticle of the nanoparticle-peptide conjugates comprises a precursor nanoparticle chemically conjugated to a plurality of polyethylene glycol (PEG) molecules. Non-limiting examples PEG molecules are selected from: SM(PEG)₂, SM(PEG)₄, SM(PEG)₆, or SM(PEG)₈.

As used herein, the term “precursor nanoparticle” refers to a nanoparticle that has been conjugated to PEG molecules, but not yet conjugated to biomimetic peptides. Non-limiting examples of such precursor nanoparticles include, for example, a spherical, virus-like particle (VLP) or a rod-shaped virus nanoparticle (VNP) or capsid proteins (CPs) or mutants thereof derived from each. Additional examples include for example, a virus-based nanoparticle is selected from a tobacco mosaic virus, a virus from the Virgaviridae family, a virus from the family Potyviridae, a virus from the family Potato virus X, a virus from the Pepino mosaic virus, a virus from the family Alphaflexiviridae, or a mutant of each thereof. As is apparent to the skilled artisan, the nanoparticle of the precursor will also define the nanoparticle element of the conjugate.

In one aspect, the precursor or virus-based nanoparticle is a not a spherical nanoparticle but rather a rod-shaped nanoparticle or capsid protein. As used herein, the term “rod-shaped” intends a shape with an aspect ratio of greater than 1. Non-limiting examples of such include a VLP from bacteriophage Qβ (Qβ) or an equivalent thereof and a VNP from a TMV or a TMV-Lys (also referred to herein as “TMV”) or an equivalent of each thereof. TMV-Lys is a variant of the wild-type TMV which has an alanine/threonine to lysine mutation at position 158 in the coat protein that grants conjugations functionality. In some aspects TMV-Lys is identified herein as “TMV.”

In another aspect, the ApoAI peptide of the conjugate or precursor is a full-length ApoAI peptide or a fragment selected from: 4FN (SEQ ID NO: 1), 4FC (SEQ ID NO: 2), or 23MJ (SEQ ID NO: 3), or an equivalent of each thereof.

In another aspect, the biomimetic ApoAI peptide is chemically conjugated to the outer surface of the nanoparticle by a maleimide-thiol reaction. In some aspects the nanoparticle, VLP or VNP is conjugated to 100-500 peptides per nanoparticle. In other aspects, the nanoparticle, VLP or VLP is conjugated to 400-600 peptides per nanoparticle. The peptides and/or VNP and/or VLP can be the same or different from each other.

In another aspect, the biomimetic ApoAI peptides include a CGGG or CSGGG linker on either the N- or the C-terminus or both.

Also provided are a plurality of conjugates and/or precursors as described herein wherein the nanoparticles or the peptides in the plurality can be the same or different from each.

In a further aspect, the conjugates or precursors are detectably labeled.

In another aspect the conjugates and/or precursors in the plurality can be detectably labeled.

Further provided are compositions comprising, or consisting essentially of, or yet further consisting of a conjugate or precursor and/or a plurality as disclosed herein and a carrier, optionally a pharmaceutically acceptable carrier. The conjugates or precursors in the composition can be the same or different from each other. The compositions can include additional therapeutic agents that are useful for their designated purpose, e.g., the treatment of CVD and associated disorders.

The conjugates and compositions are useful in vitro and in vivo. In one aspect, the conjugates and compositions are useful to deliver a biomimetic ApoAI peptide to a cell or tissue by contacting the cell or tissue with the conjugate or composition. The contacting can be in vitro or in vivo. When performed in vitro, the methods are useful for functional assays and to test new combination or personalized therapies. When performed in vivo in an animal model, the methods are useful to treat the animal or for use in functional or personalized therapies.

Further provided are methods for one or more of the following: increasing cholesterol efflux in a subject, reducing reactive oxygen species in a subject, increasing endothelial cell healing rate in a subject, ameliorating an atherosclerosis disease state in a subject, or ameliorating a cardiovascular disease state in a subject, the methods comprising, or consisting essentially of, or yet further consisting of administering a conjugate, the plurality or composition as disclosed here to the subject in need thereof, thereby providing one or more of: increasing cholesterol efflux in a subject, reducing reactive oxygen species in a subject, increasing endothelial cell healing rate in a subject, ameliorating an atherosclerosis disease state in a subject, or ameliorating a cardiovascular disease state in a subject.

Modes of administration are known in the art and described herein. Treatment includes restorative as well as preventative therapy. In one aspect, the treatment is restorative therapy only.

Further provided are methods for treating one or more of atherosclerosis, cardiovascular disease, or unsafe LDL cholesterol levels in a subject in need thereof, comprising, or consisting essentially of, or yet further consisting of, administering to the subject in need thereof of an effective amount of the multivalent nanoparticle-peptide conjugates of this disclosure, or the plurality as disclosed herein, or the composition as disclosed herein, thereby providing one or more of treating one or more of atherosclerosis, cardiovascular disease, or unsafe LDL cholesterol levels.

Modes of administration are known in the art and described herein. Treatment includes restorative as well as preventative therapy. In one aspect, the treatment is restorative therapy only.

The methods can further comprise an administration of another therapy for the treatment of the underlying disorder, e.g., administration of a statin.

The term “subject” includes mammals and human patients. In one aspect, the human patient has one or more of the following conditions: atherosclerosis, cardiovascular disease, or unsafe LDL cholesterol levels.

Further provided are kits comprising one or more conjugates, pluralities, or compositions as disclosed herein, and optionally instructions for use.

The conjugates and compositions are also useful outside of medicine, such as in the food industry as TMV-ApoAI could be a useful cholesterol-binder in raw milk filtration. Thus, in one aspect provided herein is a method to bind cholesterol in a foodstuff by contacting the foodstuff with a conjugate or composition of this disclosure. The conjugate bound to the cholesterol is then isolated from mixture.

### BRIEF DESCRIPTION OF THE DRAWINGS

FIGS. 1A-1B: Preparation of TMV-ApoAI and Qβ-ApoAI conjugates. (FIG. 1A) Biomimetic ApoAI peptides. (FIG. 1B) Bioconjugation of ApoAI peptides to TMV and Qβ using maleimide-thiol chemistry. TMV and Qβ were modeled in Chimera software (PDB ID: 2TMV and 1QBE, respectively). VLPs/VNPs and peptides are not drawn to scale.

FIGS. 2A-2F: Characterization of TMV-ApoAI peptide conjugates. (FIG. 2A) SDS-PAGE with Coomassie Blue (CB) stain: lane L (ladder), lane 1 unmodified TMV (TMV), lane 2 T-4FN conjugate (T-4FN), lane 3 TMV-4FC conjugate (T-4FC), lane 4 TMV-23MJ conjugate (T-23MJ). (FIG. 2B) Bioanalyzer (BA) electrophoretic analysis. (FIG. 2C) Summarized degree of modification. (FIG. 2D) Dynamic light scattering intensity plot of TMV-ApoAI peptide conjugates (Zavg data are mean values, n=3). (FIG. 2E) Fast protein liquid chromatography (FPLC) of TMV nanoparticles; 260:280 nm ratio at max absorption is ˜1.2. (FIG. 2F) TEM images of negatively stained TMV-ApoAI peptide conjugates (scale bars=100 nm).

FIGS. 3A-3C: Characterization of Qβ-4FN. (FIG. 3A) SDS-PAGE: lane L (ladder), lane 1 unmodified Qβ, lane 2 Qβ-4FN. (FIG. 3B) Dynamic light scattering intensity plots of Qβ and Qβ-4FN (Zavg data are mean values, n=3). (FIG. 3C) TEM images of negatively stained Qβ and Qβ-4FN (scale bars=100 nm).

FIGS. 4A-4F: Cholesterol efflux is improved by the multivalent display of ApoAI peptides on TMV and Qβ. (FIG. 4A) Schematic representation of the cholesterol efflux assay sequence. (FIG. 4B) Cholesterol efflux under upregulated ABCA1 conditions. Cholesterol efflux (%)=supernatant cholesterol/(supernatant cholesterol+cell lysate cholesterol)×100. (FIG. 4C) Bar graph shows the efflux comparison between TMV-ApoAI conjugates and HDL (LO=particle concentration of 5.5 nM, HI=particle concentration of 0.5 μM). (FIG. 4D) Cholesterol efflux under homeostatic ABCA1 conditions. (FIG. 4E) Cholesterol efflux under homeostatic ABCA1 conditions following HI treatment with the free peptide corresponding to the peptide displayed on conjugates. (FIG. 4F) Bar graph shows the efflux comparison between TMV-ApoAI conjugates and corresponding free peptides at the HI particle concentration. Data are mean fluorescence intensity ±standard deviations (n=3). Statistical analysis by one-way ANOVA with Tukey's test (*p<0.05, **p<0.01, ***p<0.001, ****p<0.0001, ns=not significant).

FIGS. 5A-5D: Cholesterol efflux measured by confocal fluorescence microscopy (panels FIG. 5A and FIG. 5B). Washed and fixed J774 foam cells post-12 hr treatment period: PBS only, 0.5 μM TMV-5FAM in PBS (control), and 0.5 μM T-4FN-5FAM in PBS (candidate). Green=TMV or T-4FN particles labeled with 5FAM. Red=Dil-Ac-LDL cholesterol. Blue=nuclei stained with 10 g/mL DAPI. Purple=cell membrane stained with wheat-germ agglutinin AlexaFluor647. Scale bars=30 μm. Panel B shows a single cell 3D reconstruction of cells after T-4FN-5FAM and TMV-5FAM treatment. (FIG. 5C) Comparison between treatment groups of total fluorescent intensity of cell-associated Dil-Ac-LDL after incubation and (FIG. 5D) corresponding total fluorescence intensity of cell-associated 5FAM after incubation. Data in (FIG. 5C) and (FIG. 5D) are means±standard deviation of n=30 (10 cells quantified per image, three images per treatment group). Statistical analysis by one-way ANOVA with Tukey's test (*p<0.05, **p<0.01, ***p<0.001, ****p<0.0001, ns=not significant).

FIGS. 6A-6B: Free BODIPY-cholesterol binding assay. (FIG. 6A) Dynamic light scattering intensity plots of TMV-ApoAI peptide conjugates before (black) and after (blue) incubation in vitro with BODIPY-cholesterol. (FIG. 6B) Fluorescence analysis of BODIPY-cholesterol binding to nanoparticles. Data are mean changes in fluorescence ±standard deviations (n=3). Statistical analysis by one-way ANOVA with Tukey's test (*p<0.05, **p<0.01, ***p<0.001, ****p<0.0001, ns=not significant).

FIGS. 7A-7B: ROS levels were detected in SVEC cells using a 2′,7′-dichlorofluorescein diacetate fluorescent probe followed by confocal fluorescence microscopy after treatment for 8 h with the VNPs and controls followed by exposure to oxidized-LDL for 4 h. (FIG. 7A) Representative image of two images captured from each sample well. (FIG. 7B) Fluorescent integrated density of intracellular ROS quantified using ImageJ. Data are mean values from the two images captured per well.

FIGS. 8A-8B: Wound healing assay. Cells were treated with 0.5 μM HDL, 4FN, T-4FN or unmodified TMV followed by 4-h exposure to 50 μg/mL ox-LDL to induce oxidative stress before monitoring wound healing for 12 (FIG. 8A) or 24 h (FIG. 8B). Data are mean changes in fluorescence ±standard deviations (n=3). Statistical analysis by one-way ANOVA with Tukey's test (*p<0.05, **p<0.01, ***p<0.001, ****p<0.0001, ns=not significant). **Note that description indicates 8-hour exposure—please clarify. “Applicant compared the effects of 8-h treatments with 0.5 μM HDL, 4FN, T-4FN or unmodified TMV followed by 4-h exposure to 50 μg/mL ox-LDL on wound closure for 12 and 24 h following 8-h treatments.”

FIGS. 9A-9B: (FIG. 9A) Apolipoprotein AI (ApoAI) monomer, dimer, and dimer hydrophobicity were modeled using Chimera (PDB ID: 2AV1monomer and 1A01 dimer). Class A and Class Y α-helices are shown in the structure. (FIG. 9B) Helical wheel analysis of ApoAI native peptide segments (helices 7 and 10) as well as the corresponding engineered biomimetic peptides. Helical wheels were generated using CPT Galaxy-Apollo. Hydrophobic faces and vector moments were determined using Heliquest. The ApoAI protein and peptides are not shown to scale.

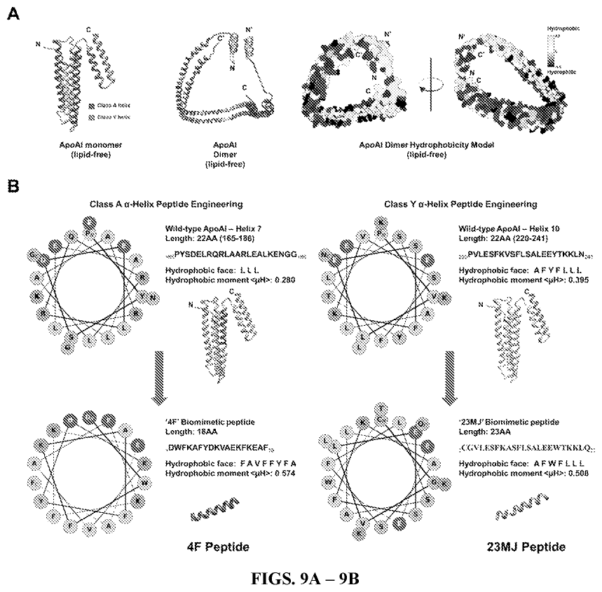

FIGS. 10A-10E: Synthesis and characterization of TMV-5FAM. (FIG. 10A) Conjugation of 5FAM to internal GLU97 & 106 residues of TMV. (FIG. 10B) SDS-PAGE: lane L (ladder), lane 1 TMV-5FAM, lane 2 T4FN-5FAM. (FIG. 10C) Dynamic light scattering intensity plots of TMV particles (Zavg data are mean values ±SD, n=3). (FIG. 10D) UV/vis absorbance of TMV particles. (FIG. 10E) Corresponding TEM images of negatively stained TMV conjugates (scale bars represent 100 nm).

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral is found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

The term “biomimetic” refers to a bioengineering strategy that involves emulating naturally occurring systems, structures, or molecules when creating solutions to complex human biological problems. Biomimetic approaches take advantage of millions of years of natural selection that has given rise to numerous naturally occurring solutions to various challenges. Exemplary challenges include but are not limited to self-healing abilities, environmental exposure tolerance and resistance, hydrophobicity, self-assembly, and harnessing solar energy.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally occurring or non-naturally occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs, and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

An “effective amount” or “efficacious amount” refers to the amount of an agent or combined amounts of two or more agents, that, when administered for the treatment of a mammal or other subject, is sufficient to affect such treatment for the disease. The “effective amount” will vary depending on the agent(s), the disease and its severity and the age, weight, etc., of the subject to be treated. In some embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

Cardiovascular disease (CVD) is a general term for conditions affecting the heart or blood vessels. It's usually associated with a build-up of fatty deposits inside the arteries (atherosclerosis) and an increased risk of blood clots. It can also be associated with damage to arteries in organs such as the brain, heart, kidneys and eyes. As used herein, CVD intents subjects with active disease or at high risk of such disease.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, the term “administer” or “administration” or “administering” intends to mean delivery of a substance to a subject such as an animal or human. Administration can be accomplished in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, and inhalation.

An agent of the present disclosure can be administered for therapy by any suitable route of administration. It will also be appreciated that the optimal route will vary with the condition and age of the recipient, and the disease being treated.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank +EMBL+DDBJ+PDB+GenBank CDS translations+SwissProtein+SPupdate+PIR. Details of these programs can be found at the following Internet address: ncbi.nlm.nih.gov/cgi-bin/BLAST.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term “Apolipoprotein AI” (ApoAI) refers a protein that in humans is encoded by the APOA1 gene. As the major component of HDL particles, it has a specific role in lipid metabolism. The protein, as a component of HDL particles, enables efflux of fat molecules by accepting fats from within cells (including macrophages within the walls of arteries which have become overloaded with ingested fats from oxidized LDL particles) for transport (in the water outside cells) elsewhere, including back to LDL particles or to the liver for excretion. It is a cofactor for lecithin-cholesterol acyltransferase (LCAT) which is responsible for the formation of most plasma cholesteryl esters. Apolipoprotein AI has also been isolated as a prostacyclin (PGI2) stabilizing factor, and thus may have an anticlotting effect. Defects in the gene encoding it are associated with HDL deficiencies, including Tangier disease, and with systemic non-neuropathic amyloidosis. Apo-AI is often used as a biomarker for prediction of cardiovascular diseases. The ratio apoB-100/apoA1 (i.e., LDL & larger particles vs. HDL particles), NMR measured lipoprotein (low density lipoprotein (LDL)/(HDL) particle ratios even more so, has always had a stronger correlation with myocardial infarction event rates than older methods of measuring lipid transport in the water outside cells.

As used herein, the term “aspect ratio” intends the ratio of the width to the height of the nanoparticle or peptide. Thus, an aspect ratio of greater than 1 intends that the nanoparticle or peptide is not spherical, that one dimension is longer than the other ratio.

The term “conjugation” or “bioconjugation” as used herein refers to a chemical technique used to couple two molecules together, at least one of which is a biomolecule, such as a carbohydrate, nucleic acid, or protein. Exemplary reactions include, but are not limited to, cysteine-disulfide linkages, cysteine-maleimide linkages, or lysine-isocyanate linkages.

The term “polyethylene glycol” (PEG) refers a polyether compound derived from petroleum with many applications, from industrial manufacturing to medicine. PEGs are prepared by polymerization of ethylene oxide and are commercially available over a wide range of molecular weights from 300 g/mol to 10,000,000 g/mol. Due to their variable size and inert chemical nature, PEGs have a variety of biomedical uses. These uses may include, but are not limited to: crowding agent, precipitant, or packaging vector. PEGs may also be further functionalized in a variety of ways one of which is usage as heterobifunctional crosslinkers with N-hydroxysuccinimide (NHS) ester and maleimide groups that allow covalent conjugation of amine- and sulfhydryl-containing molecule. Such functionalized PEGs can be referred to SMPEGs. SMPEGs may consists of two thiol maleimide functional groups separated by a set number of PEGs. For example, SM(PEG)₂ has the structure:

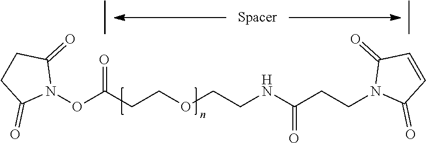

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

The term “precursor nanoparticle” as used herein refers to a peptide, a virus-based nanoparticle, VNP, VLP or equivalent thereof that has been conjugated to PEG or SMPEG molecules but has not yet been further conjugated to biomimetic ApoAI peptides.

The term “low density lipoprotein” (LDL) refers to lipoproteins that transfer lipids (fats) around the body in the extracellular fluid, making fats available to body cells for receptor-mediated endocytosis. Lipoproteins are complex particles composed of multiple proteins, typically 80-100 proteins per particle (organized by a single apolipoprotein B for LDL and the larger particles). A single LDL particle is about 220-275 angstroms in diameter, typically transporting 3,000 to 6,000 fat molecules per particle, and varying in size according to the number and mix of fat molecules contained within. The lipids carried include all fat molecules with cholesterol, phospholipids, and triglycerides dominant; amounts of each vary considerably. Of particular interest is that LDL is involved in atherosclerosis, a process in which it is oxidized within the walls of arteries.

The term “high-density lipoprotein” (HDL) refers to complex particles composed of multiple proteins which transport all fat molecules (lipids) around the body within the water outside cells. HDL's size ranges from 5 to 17 nm and HDL is the smallest of the lipoprotein particles. HDL is the densest because it contains the highest proportion of protein to lipids, and its most abundant apolipoproteins are apo A-I and apo A-II. Increasing concentrations of HDL particles are associated with decreasing accumulation of atherosclerosis within the walls of arteries, reducing the risk of sudden plaque ruptures, cardiovascular disease, stroke and other vascular diseases.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (₆′ ed.). Examples of luminescent probes include, but are not limited to, aequorin and luciferases.

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

The term nanoparticle when used in the context of this disclosure intends a virus-based or protein-based nanostructure that can be spherical or non-spherical. The nanoparticle proteins can be in one embodiment the capsid proteins of the virus-based nanoparticle. As described herein, the term intends wildtype as well a mutant structure that in one aspect, provide substantially similar function as described herein. Non-limiting examples of wherein the virus-based nanoparticle is selected from a nanoparticle from a virus selected from a tobacco mosaic virus, a virus from the Virgaviridae family, a virus from the family Potyviridae, a virus from the family Potato virus X, a virus from the Pepino mosaic virus, a virus from the family Alphaflexiviridae, or a mutant of each thereof. The virus-based nanoparticles can in one aspect by a VNP or a VLP.

Tobacco Mosaic Virus (TMV) is a member of the Virgaviridae family. This virus is among the first if not the first characterized virus and has been utilized as a model virus for decades for life science applications.

TMV has a rod-like appearance. Its capsid is made from 2130 molecules of coat protein and one molecule of genomic single strand RNA, 6400 bases long. The coat protein self-assembles into the rod-like helical structure (16.3 proteins per helix turn) around the RNA, which forms a hairpin loop structure. The protein monomer consists of 158 amino acids which are assembled into four main alpha-helices, which are joined by a prominent loop proximal to the axis of the virion. Virions are ˜300 nm in length and ˜18 nm in diameter. The RNA is located at a radius of ˜4 nm and is protected from the action of cellular enzymes by the coat protein. X-ray fiber diffraction structure of the intact virus was studied based on an electron density map at 3.6 Å resolution. Inside the capsid helix, near the core, is the coiled RNA molecule, which is made up of 6,395±10 nucleotides.

In some embodiments, a VNP derived from TMV-lys comprise, or consists essentially of, or yet further consists of, a plurality of coat proteins (CPs). In some embodiments, the coat protein is a wild-type VNP coat protein. In further embodiments, the coat protein is modified, e.g., comprising, or consisting essentially of, or yet further consisting of, one or more substitutions, insertions, and/or deletions. TMV-Lys is a mutant of TMV, and each of the 2130 coat proteins (CP) contain an A158K substitution with a one-residue deletion achieved through genetic engineering, which provides amine functional group reactivity on the external surface of the viral nanoparticle for downstream biconjugation chemistry.

In some embodiments, a TMV coat protein comprises, or alternatively consists essentially of, or yet further consists of the sequence as set forth in the UniProtKB ID P69687:
MSYSITTPSQFVFLSSAWADPIELINLCTNALGNQFQTQQARTVVQRQFSEVWKPSPQ VTVRFPDSDFKVYRYNAVLDPLVTALLGAFDTRNRIIEVENQANPTTAETLDATRRV DDATVAIRSAINNLIVELIRGTGSYNRSSFESSSGLVWTSGPAT (SEQ ID NO: 4), or
the functionalized variant TMV-LYS:
MSYSITTPSQFVFLSSAWADPIELINLCTNALGNQFQTQQARTVVQRQFSEVWKPSPQ VTVRFPDSDFKVYRYNAVLDPLVTALLGAFDTRNRIIEVENQANPTTAETLDATRRV DDATVAIRSAINNLIVELIRGTGSYNRSSFESSSGLVWTSGPK (SEQ ID NO: 5), or an equivalent thereof.

Bacteriophage Qβ (Qbeta or alternatively QB) is a member of the leviviridae family. It is a small virus that is about 25 nm thick and is a coliphage with an RNA that is 4217 nucleotides long. As described in biology.kenyon.edu/BMB/jsmol2019/EAIJ/NewVersion81.html #:˜:text=Bacteriophage %20 QB %20is %20a %20member,%2C %20%26%20Finn %2C %202009).&text=Members %20 of % 20the %201eviviridae %20family,et %20at. %2C %202018) last accessed on Aug. 17, 2021, QB has 20 faces each composed of six subunits and 12 vertices each composed of 5 subunits. Members of the leviviridae family form icosahedral capsids from 180 coat protein subunits around a 4.2 kb sense-strand RNA genome. Each of these coat proteins (capsomers) has about 132 residues of amino acids. Bacteriophage QB is a positive strand RNA virus. Positive strand RNA viruses have genomes that are functional mRNAs. For instance, QB's genome codes for 4 proteins: A1, A2, CP and QB replicase. QB has other proteins like the B-subunit of a replicase, the maturation protein A2 and a minor protein A1. The penetration of the virus into a host cell is quickly followed by translation to produce RdRps and other viral proteins that are required for the production of more viral RNAs. QB ssRNA adsorb to bacterial sex pili proteins and infect. Like other RNA viruses, QB replicates its genome by utilizing virally encoded RNA polymerase (RdRp). The genome is used as the template for the synthesis of other RNA strands. Upon infection, the B-subunit interacts with host proteins to form a complex. The complex contains RNA-helicases to unwind DNA and NTPases that are useful for polymerization. Once the complex forms, the transcription of the genome, a copy of the genome, and mRNAs begin. Phage MS2 has the same genome as QB.

Bacteriophage Qβ coat protein self-assembles to form an icosahedral capsid with a T=3 symmetry, about 26 nm in diameter, and consisting of 89 capsid proteins dimers (178 capsid proteins). It is also involved in viral genome encapsidation through the interaction between a capsid protein dimer and the multiple packaging signals present in the RNA genome. Binding of the capsid proteins to the viral RNA induces a conformational change required for efficient T=3 shell formation. Additionally, it acts as a translational repressor of viral replicase synthesis late in infection. This latter function is the result of capsid protein interaction with an RNA hairpin which contains the replicase ribosome-binding site. See, for example, Gorzelnik et al., Proc Natl Acad Sci USA. 2016 Oct. 11; 113(41):11519-11524; Basnak et al., J Mol Biol. 2010 Feb. 5; 395(5):924-36; and Lim et al., J Biol Chem. 1996 Dec. 13; 271(50):31839-45.

In some embodiments, a VLP derived from bacteriophage Qβ comprise, or consists essentially of, or yet further consists of, a plurality of coat proteins (CPs). In some embodiments, the coat protein is a wild-type bacteriophage Qβ coat protein. In further embodiments, the coat protein is modified, e.g., comprising, or consisting essentially of, or yet further consisting of, one or more substitutions, insertions, and/or deletions. In some embodiments, a bacteriophage Qβ coat protein comprises, or alternatively consists essentially of, or yet further consists of the sequence as set forth in the UniProtKB ID P03615: MAKLETVTLGNIGKDGKQTLVLNPRGVNPTNGVASLSQAGAVPALEKRVTVSVSQβ SRNRKNYKVQVKIQNPTACTANGSCDPSVTRQAYADVTFSFTQYSTDEERAFVRTEL AALLASPLLIDAIDQLNPAY (SEQ ID NO: 6) or an equivalent thereof.

A bacteriophage Q3 hairpin loop refers to a portion of a Q3 RNA where a Q3 coat protein can bind to. In some embodiments, the hairpin loop serves as a packaging signal directing an RNA comprising the hairpin loop to be encapsidated in a capsid comprising, or consisting essentially of, or yet further consisting of a Qβ coat protein.

Proteinaceous viral nanoparticles (VNPs) were employed as templates to synthesize biomimics of high-density lipoprotein (HDL) that achieve multivalent display of a library of ApoAI peptides through covalent surface chemistry. S Bioconjugation yielded a biomimic library of VNP-ApoAI conjugates—derived from bioinspired nanocarriers such as plant virus and bacteriophage—with varying size and morphology. VNP-ApoAI conjugates were evaluated through several functional in vitro assays: ABCA1-mediated cholesterol efflux of using macrophage foam cells, mitigation of ROS in endothelial cells, and wound healing in endothelial cells. Results from these studies demonstrate that multivalent VNP-ApoAI significantly improves therapeutic efficacy over free peptide formulations and traditional HDL therapy. Finally, to better understand the mechanistic basis of the observed cholesterol efflux enhancement, biomimics undergoing cholesterol efflux were studied under confocal fluorescent microscopy and noted to primarily localize on the exterior of foam cell membrane surfaces. Building on this research, this disclosure provides a multivalent nanoparticle-peptide conjugate that reduces low-density lipoprotein (LDL)-cholesterol in a subject, thereby ameliorating atherosclerosis disease states and cardiovascular disease states. Applicant chemically conjugated biomimetic ApoAI peptides to nanoparticles such as bacteriophage Qβ (QP) and TMV-Lys (TMV). Applicant used functional polyethylene glycol to link the ApoAI peptides to the target nanoparticles.

### Virus-Like Particles (VLPs) and Rod-Shaped Virus Nanoparticle (VNPs)

VLPs and VNPs are generally composed of one or more viral proteins, such as, but not limited to, those proteins referred to as capsid, coat, shell, surface and/or envelope proteins, or particle-forming polypeptides derived from these proteins. VLPs and VNPs can form spontaneously upon recombinant expression of the protein in an appropriate expression system. VLPs and VNPs can also be engineered, e.g., comprising, or consisting essentially of, or yet further consisting of, one or more viral proteins that comprise, or consists essentially of, or yet further consists of, a modification. Methods for producing VLPs and VNPs are known in the art. The presence of VLPs and VNPs following recombinant expression of viral proteins can be detected using conventional techniques known in the art, such as by electron microscopy, biophysical characterization, and the like. Further, VLPs and VNPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354: 53073, 2012).

Provided herein are multivalent nanoparticle-peptide conjugate, comprising one or more of a biomimetic ApoAI peptide chemically conjugated to an outer surface of a nanoparticle. In some aspects, the VLP is bacteriophage Qβ (Qβ) and the VNP is TMV-Lys (TMV).

In one aspect, the nanoparticle comprises a precursor nanoparticle chemically conjugated to a plurality of polyethylene glycol (PEG) molecules. Non-limiting examples of PEG molecules are selected from: SM(PEG)₂, SM(PEG)₄, SM(PEG)₆, or SM(PEG)₈. In some embodiments, the precursor nanoparticle is selected from a spherical, virus-like particle (VLP) or a rod-shaped virus nanoparticle (VNP). Non-limiting examples of ApoAI peptides are selected from: 4FN (SEQ ID NO: 1), 4FC (SEQ ID NO: 2), or 23MJ (SEQ ID NO: 3), or an equivalent of each thereof. In some aspects, the one or more biomimetic ApoAI peptides are chemically conjugated to the outer surface of the nanoparticle by a maleimide-thiol reaction.

In several embodiments, the VLP of the conjugate is QB and the ApoAI peptide is selected from 4FN (SEQ ID NO: 1), 4FC (SEQ ID NO: 2), or 23MJ (SEQ ID NO: 3), or an equivalent of each thereof, that optionally further comprise PEG molecules that are selected from SM(PEG)₂, SM(PEG)₄, SM(PEG)₆, or SM(PEG)₈.

In other aspects, the VLP of the conjugate is TMV-Lys (TMV) and the ApoAI peptide is selected from 4FN (SEQ ID NO: 1), 4FC (SEQ ID NO: 2), or 23MJ (SEQ ID NO: 3), or an equivalent of each thereof, that optionally further comprise PEG molecules that are selected from SM(PEG)₂, SM(PEG)₄, SM(PEG)₆, or SM(PEG)₈.

Also provided are a plurality of multivalent nanoparticle-peptide conjugates as described herein, wherein the plurality are the same of different from each other.

In some embodiments, the virus, or VNP is derived from Tobaccos Mosaic Virus (TMV). In some embodiments, the coat protein of TMV of the conjugate comprises, or alternatively consists essentially of, or yet further consists of the sequence as set forth in the UniProtKB ID: P69687:
MSYSITTPSQFVFLSSAWADPIELINLCTNALGNQFQTQQARTVVQRQFSEVWKPSPQ VTVRFPDSDFKVYRYNAVLDPLVTALLGAFDTRNRIIEVENQANPTTAETLDATRRV DDATVAIRSAINNLIVELIRGTGSYNRSSFESSSGLVWTSGPAT (SEQ ID NO: 4), or
the functionalized variant TMV-LYS:
MSYSITTPSQFVFLSSAWADPIELINLCTNALGNQFQTQQARTVVQRQFSEVWKPSPQ VTVRFPDSDFKVYRYNAVLDPLVTALLGAFDTRNRIIEVENQANPTTAETLDATRRV DDATVAIRSAINNLIVELIRGTGSYNRSSFESSSGLVWTSGPK (SEQ ID NO: 5), or an equivalent thereof.

TMV-Lys is a mutant of TMV, and each of the 2130 coat proteins (CP) contain a T158K substitution achieved through genetic engineering, which provides amine functional group reactivity on the external surface of the viral nanoparticle for downstream bioconjugation chemistry.

In some aspects the VLP is or is derived from Bacteriophage Qβ (Qbeta or alternatively Qbeta bacteriophage) which is a member of the levivirida family.

In some embodiments, a VLP derived from bacteriophage Qβ comprise, or consists essentially of, or yet further consists of, a plurality of coat proteins. In some embodiments, the coat protein is a wild-type bacteriophage Qβ coat protein. In further embodiments, the coat protein is modified, e.g., comprising, or consisting essentially of, or yet further consisting of, one or more substitutions, insertions, and/or deletions. In some embodiments, a bacteriophage Qβ coat protein comprises, or alternatively consists essentially of, or yet further consists of the sequence as set forth in the UniProtKB ID P03615:
MAKLETVTLGNIGKDGKQTLVLNPRGVNPTNGVASL SQAGAVPALEKRVTVSVSQP SRNRKNYKVQVKIQNPTACTANGSCDPSVTRQAYADVTF SFTQYSTDEERAFVRTEL AALLASPLLIDAIDQLNPAY (SEQ ID NO: 6) or an equivalent thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a virus or VLP.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid. Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some instances, a virus or VLP described herein further comprise, or consists essentially of, or yet further consists of, a label or a tag, e.g., such as a detectable label. A detectable label can be attached to, e.g., to the surface of a virus or VLP.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As, ⁷⁶Br, ⁷⁷Br, ^{81m}Kr, ⁸²Rb, ⁸⁵Sr, ⁸⁹Sr, ⁸⁶Y, ⁹⁰Y, ⁹⁵No, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴I, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe (III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the virus or VLP or nanoparticle. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

The proteins, VNP, VLP and/or capsid can be detectably labeled.

Methods to make the conjugates and precursors are provided herein, and in particular with respect to QB and TMV (TMV-Lys). The disclosed methods can be modified by one of skill in the art to make and use all the conjugates and precursors within the scope of the appended claims.

### Compositions

In another aspect, provided herein is a composition comprising, consisting essentially of, or consisting of the combination of formulations comprising a multivalent nanoparticle-peptide conjugate as provided herein, and at least one carrier, such as a pharmaceutically acceptable carrier or excipient. In another aspect, a plurality of the multivalent nanoparticle-peptide conjugates are provided herein, wherein the nanoparticle-peptide conjugates can be the same or different from each other.

In one aspect, the composition further comprises a preservative or stabilizer, that can be in one aspect, exogenously added or non-naturally occurring.

In one embodiment, this technology relates to a composition comprising a combination of multivalent nanoparticle-peptide conjugate or formulations as described herein and a carrier.

In another embodiment, this technology relates to a pharmaceutical composition comprising a combination of multivalent nanoparticle-peptide conjugate or formulations as described herein and a pharmaceutically acceptable carrier.

In another embodiment, this technology relates to a pharmaceutical composition comprising an effective amount or a therapeutically effective amount of a multivalent nanoparticle-peptide conjugate as described herein and a pharmaceutically acceptable carrier.

Compositions, including pharmaceutical compositions comprising, consisting essentially of, or consisting of the multivalent nanoparticle-peptide conjugate formulation alone or in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, oral, buccal, rectal, sublingual, or transdermal administration routes. In some cases, parenteral administration comprises, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins, 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL©, dibasic calcium phosphate, dicalcium phosphate dihydrate, tricalcium phosphate, calcium phosphate, anhydrous lactose, spray-dried lactose, pregelatinized starch, compressible sugar, such as Di-PAC© (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL©, or sodium starch glycolate such as PROMOGEL© or EXPLOTAB©, a cellulose such as a wood product, methylcrystalline cellulose, e.g., AVICEL©, AVICEL© PH101, AVICEL*PH102, AVICEL© PH105, ELCEMA© P100, EMCOCEL©, VIVACEL©, MING TIA©, and SOLKA-FLOC©, methylcellulose, croscarmellose, or a cross-linked cellulose, such as cross-linked sodium carboxymethylcellulose (AC-DI-SOL©), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM© HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX©), higher fatty acids and their alkali-metal and alkaline earth metal salts, such as aluminum, calcium, magnesium, zinc, stearic acid, sodium stearates, glycerol, talc, waxes, STEAROWET©, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL©, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC© (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Atherosclerosis and Cardiovascular Applications

Atherosclerosis is a progressive cardiovascular disease (CVD) in which excess cholesterol accrues within the arterial intima as a fibrofatty plaque.¹ In late stage CVD, thrombosis and plaque rupture can occur, resulting in myocardial infarction or stroke, which are the two leading causes of death worldwide.² Moreover, patients who recovered from thrombotic complication or stroke can suffer long-term consequence; the degree and permanence of heart, brain, or organ damage is directly related to the duration of insufficient oxygen supply.

The molecular basis of atherosclerosis progression is still under investigation; however, atherosclerosis has been framed as an inflammatory disease where genetic predisposition and lifestyle fosters inflammation which triggers endothelial cell dysfunction, proteoglycan remodeling, and a homeostatic imbalance between cholesterol deposition into and cholesterol removal or “efflux” from the arteries.^{3,4,5} Cholesterol is delivered into cells mainly by a biocarrier known as low-density lipoprotein (LDL) via LDL receptor-mediated endocytosis. In contrast, the extraction of excess cholesterol from cells to waste streams for excretion from the body is accomplished via biocarrier high-density lipoprotein (HDL); a major protein within HDL is Apolipoprotein A1 (ApoA1) which interacts with ATP-binding cassette transporter A1 (ABCA1) and scavenger-receptor class B type I (SR-BI) to facilitate cholesterol efflux from fatty macrophages known as foam cells.⁶ Reverse cholesterol transport and cholesterol efflux function are understood to be the primary mechanisms by which high-density lipoprotein (HDL) removes excess cholesterol removal from the body and cells, respectively. An inverse relationship between HDL and risk of CVD is well-documented.⁶ The current treatment landscape for atherosclerosis includes preemptive and reactive interventions such as lifestyle changes, medicine, and surgery to manage healthy levels of cholesterol in the body—cholesterol-lowering medications are highlighted below.

Clinically approved statins focus on mitigating lipids and LDL-cholesterol or “bad” cholesterol through an indirect mechanism approach, which involves blocking cholesterol production in the body through enzyme inactivation. Consequently, it is hypothesized that lowering cholesterol levels in circulating blood promotes cholesterol efflux from LDL cholesterol-rich plaques in the arteries. However, an unfortunate side effect from long-term statin use is the upregulation of proprotein convertase subtilisin/kexin type 9 serine protease (PCSK9), an inhibitor of LDL receptor—a receptor in the liver purposed for cholesterol excretion from the body, which presents a paradox.⁷ Notably, the FDA recently approved an siRNA gene therapy against PCSK9 which results in LDL-cholesterol lowering.^{8,9} Bile acid sequestrants for cholesterol are an alternative to statins and have a complementary mechanism of action that stack onto the LDL-lowering effect of statins, however, suffer low patient compliance due to adverse side effects.¹⁰

Clinically tested therapies against atherosclerosis also leverage the act of mitigating LDL-cholesterol through a direct mechanism approach, which involves using HDL as a cholesterol efflux-promoting “sponge” that absorbs LDL-cholesterol by interacting with resident foam cell receptors within fibrofatty plaque. At the time, an inverse relationship between HDL concentration and CVD severity was noted. Therefore, HDL was used in clinical trials and formulated by combining serum derived ApoAI with different phospholipid compositions, reviewed elsewhere. Results were modest and difficult to reproduce, however, clinical trials were small scale.^{6, 11, 12} It has been suggested that one possible reason for its failure was the heterogeneous nature of serum-derived ApoAI from HDL donors, which may have been dysfunctional in “quality”.¹³ In fact, HDL derived from CVD patients display decreased cholesterol efflux, which was associated with an oxidized ApoAI protein and lipidome.^{14,15, 16} Interestingly, more recent clinical studies have indicated that the relationship between HDL concentration and CVD mortality is U-shaped and not linear. ^{17,18} Therefore, clinical studies and the scientific in vitro, in vivo, and ex vivo functional assays all suggest that HDL “quality”, not “quantity” is critical to lower LDL-cholesterol and combat CVD.

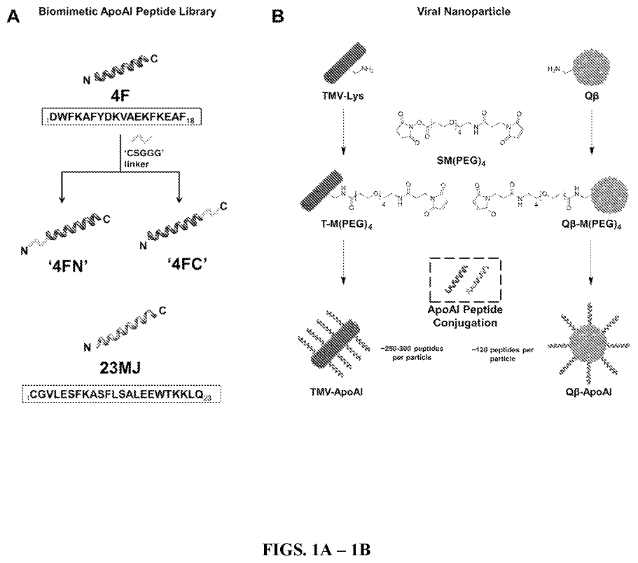

In order to generate HDL biomimetics with known quality, recombinant ApoAI was engineered and employed in clinical HDL formulations, albeit limited to small trials due to the nature of scalability, sterility, and bioavailability of hydrophobic, full-length 267AA ApoAI.^{19,20,21} Ergo, smaller biomimetic peptides of ApoAI were developed and are reviewed elsewhere.^{20,22,23, 24} These contain an amphiphilic α-helix that is functionally involved in lipid binding and cholesterol efflux. Because native ApoAI structure consists of 10 amphiphilic α-helices, ApoAI peptides have also been re-engineered into multimeric forms as tandem or branched arrangements of peptides to improve biomimetic function such as mature HDL remodeling and enhanced cholesterol efflux capacity. ApoAI peptides have previously been delivered standalone or as lipid nanoparticles formulated using dimyristyl phosphocholine (DMPC).^{25, 26} Specifically, ApoAI peptide “4F” has been clinically tested in Phase I, and while safe, bioavailability remains an issue (FIGS. 1A-1B).^{27, 28} Work spanning several decades of study has traditionally focused on the engineering ApoAI, however, its formulation has been limited to lipid nanoparticles. Recent work in the field combines lipids with inorganic and nucleic acid-based nanoparticles to load ApoAI for targeted delivery.^{29, 30, 31, 32} However, to the best of Applicant's knowledge, ApoAI peptides have not been covalently conjugated to a nanoparticle surface for multivalent display.

The present disclosure provides and utilized as an example a library of viral nanocarriers of varying shape and morphology; a 30 nm spherical virus-like particle (VLP) with T3 symmetry—bacteriophage Qβ (Qβ)—and a rod-shaped plant virus nanoparticle (VNP) with high aspect ratio shape of dimensions 300×18 nm—tobacco mosaic virus (TMV) (Table 2). TMV is an elegantly-complex model nanoparticle—composed of 2130 repeating units of coat protein which precisely self-assemble around a single piece of (+) ss-RNA. Qβ is expressed and assembled through bacterial fermentation; each particle is composed of 180 identical units of coat protein. For the present disclosure, tobacco mosaic virus mutant with lysine (TMV) substituted at position 158 after truncation to 158 amino acids (SEQ ID NO: 5) and also Qβ for its precise surface amine-enabled (SEQ ID NO: 6) are utilized for bioconjugation to a library of biomimetic ApoAI peptides: 4FN and 4FC (based on clinical 4F sequence) as well as 23MJ (based on previous preclinical multivalent efficacy data) (Table 2). It has been demonstrated that multivalent peptide display on TMV particle improves targeting to enhance atherosclerotic plaque imaging. Moreover, TMV has favorable flow and margination properties allowing the targeting of the diseased vessel wall. Therefore, TMV was leveraged to probe the functional effects of multivalent ApoAI peptide display on cholesterol efflux. Additionally, to better understand the functional implications of nanocarrier shape on multivalent peptide display-associated cholesterol efflux, Qβ-ApoAI peptide conjugates were generated as a comparator to TMV-ApoAI.

The mechanistic and functional quality of TMV-ApoAI conjugates via cholesterol binding assay, cholesterol efflux assay, ROS assay, and wound healing assay were all evaluated as part of this disclosure.

In some embodiments, the present methods comprise, or consist essentially of, or yet further consist of administering one or more of the multivalent nanoparticle-peptide conjugates, or the pluralities or the compositions as described herein. In some embodiments, the nanoparticle of the conjugate comprises a spherical, virus-like particle (VLP) or a rod-shaped virus nanoparticle (VNP). In some embodiments, the peptides conjugated to the nanoparticle comprise biomimetic ApoAI peptides. In some embodiments, the biomimetic ApoAI peptides are selected from: 4FN (SEQ ID NO: 1), 4FC (SEQ ID NO: 2), or 23MJ (SEQ ID NO: 3), or equivalents of each thereof.

In some embodiments, the nanoparticle is selected from a spherical, virus-like particle (VLP) or a rod-shaped virus nanoparticle (VNP). In some embodiments, the VLP may be a bacteriophage Qβ (Qβ) and in still other embodiments, the VNP may be TMV-Lys (TMV).

In some embodiments, functionalized polyethylene glycol (PEG) molecules may be conjugated to the outer surface of the selected nanoparticle. In some embodiments, the PEG molecules may be selected from: SM(PEG)₂, SM(PEG)₄, SM(PEG)₆, or SM(PEG)₈. In some embodiments, the result of the nanoparticle-PEG conjugation reaction comprises a precursor or intermediate molecule. In some embodiments, the conjugation reaction comprises maleimide-thiol chemistry. This chemistry can be applied to any of the proteins or virus-based nanoparticles as disclosed herein.

In some embodiments, the final multivalent nanoparticle peptide conjugate is generated by conjugating biomimetic ApoAI peptides to a previously generated nanoparticle-PEG precursor molecule. In some embodiments, the ApoAI peptides are selected from: 4FN (SEQ ID NO: 1), 4FC (SEQ ID NO: 2), or 23MJ (SEQ ID NO: 3), or an equivalent of each thereof. In some embodiments, the conjugation reaction comprises maleimide-thiol chemistry.

A plurality of ApoAI peptides can be conjugated, that may be the same or different from each other.

Further embodiments comprise a method of accomplishing one or more of the following by administering to a subject the presently described multivalent nanoparticle peptide conjugate: increasing cholesterol efflux in a subject, reducing reactive oxygen species in a subject, increasing endothelial cell healing rate in a subject, ameliorating an atherosclerosis disease state in a subject, or ameliorating a cardiovascular disease state in a subject. In some embodiments, the subject being treated has one or more of the following conditions: atherosclerosis, cardiovascular disease, or unsafe LDL cholesterol levels. Methods to detect or determine treatment status are known in the art.

Non-limiting examples of such the nanoparticle of the conjugate or composition include, for example, a spherical, virus-like particle (VLP) or a rod-shaped virus nanoparticle (VNP) or capsid proteins (CPs) or mutants thereof derived from each. Additional examples include for example, a virus-based nanoparticle is selected from a tobacco mosaic virus, a virus from the Virgaviridae family, a virus from the family Potyviridae, a virus from the family Potato virus X, a virus from the Pepino mosaic virus, a virus from the family Alphaflexiviridae, or a mutant of each thereof.

In one aspect, the virus-based nanoparticle is a not a spherical nanoparticle but rather a rod-shaped nanoparticle or capsid protein. As used herein, the term “rod-shaped” intends a shape with an aspect ratio of greater than 1. Non-limiting examples of such include a VLP from bacteriophage Qβ (QP) or an equivalent thereof and a VNP from a TMV or a TMV-Lys (also referred to herein as “TMV”) or an equivalent of each thereof. TMV-Lys is a variant of the wild-type TMV which has an alanine/threonine to lysine mutation at position 158 in the coat protein that grants conjugations functionality. In some aspects TMV-Lys is identified herein as “TMV.”

In another aspect, the ApoAI peptide of the conjugate is a full-length ApoAI peptide or a fragment selected from: 4FN (SEQ ID NO: 1), 4FC (SEQ ID NO: 2), or 23MJ (SEQ ID NO: 3), or an equivalent of each thereof.

In another aspect, the ApoAI peptide is a full-length ApoAI peptide or a fragment selected from: 4FN (SEQ ID NO: 1), 4FC (SEQ ID NO: 2), or 23MJ (SEQ ID NO: 3), or an equivalent of each thereof and the VLP is a QB or a TMV VLP or a mutant or equivalent of each thereof.

In another aspect, the biomimetic ApoAI peptide is chemically conjugated to the outer surface of the nanoparticle by a maleimide-thiol reaction. In some aspects the nanoparticle, VLP or VNP is conjugated to 100-500 peptides per nanoparticle. In other aspects, the nanoparticle, VLP or VLP is conjugated to 400-600 peptides per nanoparticle. The peptides and/or VNP and/or VLP can be the same or different from each other.

In another aspect, the biomimetic ApoAI peptides include a CGGG or CSGGG linker on either the N- or the C-terminus or both.

In one aspect, a plurality of conjugates as described herein are administered wherein the nanoparticles in the plurality can be the same or different from each.

In a further aspect, the conjugates or precursors are detectably labeled.

Modes of administration are known in the art and described herein. Treatment includes restorative as well as preventative therapy. In one aspect, the treatment is restorative therapy only.

The methods can further comprise an administration of another therapy for the treatment of the underlying disorder, e.g., administration of a statin.

The term “subject” includes mammals and human patients. In one aspect, the human patient has one or more of the following conditions: atherosclerosis, cardiovascular disease, or unsafe LDL cholesterol levels.

Also provided are methods for treating one or more of atherosclerosis, cardiovascular disease, or unsafe LDL cholesterol levels in a subject in need thereof, comprising administering to the subject in need thereof of an effective amount of the multivalent nanoparticle-peptide conjugate or the composition as disclosed herein, thereby providing one or more of treating one or more of atherosclerosis, cardiovascular disease, or unsafe LDL cholesterol levels.

Non-limiting examples of such the nanoparticle of the conjugate or composition include, for example, a spherical, virus-like particle (VLP) or a rod-shaped virus nanoparticle (VNP) or capsid proteins (CPs) or mutants thereof derived from each. Additional examples include for example, a virus-based nanoparticle is selected from a tobacco mosaic virus, a virus from the Virgaviridae family, a virus from the family Potyviridae, a virus from the family Potato virus X, a virus from the Pepino mosaic virus, a virus from the family Alphaflexiviridae, or a mutant of each thereof.

In one aspect, the virus-based nanoparticle is a not a spherical nanoparticle but rather a rod-shaped nanoparticle or capsid protein. As used herein, the term “rod-shaped” intends a shape with an aspect ratio of greater than 1. Non-limiting examples of such include a VLP from bacteriophage Qβ (Qβ) or an equivalent thereof and a VNP from a TMV or a TMV-Lys (also referred to herein as “TMV”) or an equivalent of each thereof. TMV-Lys is a variant of the wild-type TMV which has an alanine/threonine to lysine mutation at position 158 in the coat protein that grants conjugations functionality. In some aspects TMV-Lys is identified herein as “TMV.”

In another aspect, the ApoAI peptide of the conjugate is a full-length ApoAI peptide or a fragment selected from: 4FN (SEQ ID NO: 1), 4FC (SEQ ID NO: 2), or 23MJ (SEQ ID NO: 3), or an equivalent of each thereof.

In another aspect, the ApoAI peptide is a full-length ApoAI peptide or a fragment selected from: 4FN (SEQ ID NO: 1), 4FC (SEQ ID NO: 2), or 23MJ (SEQ ID NO: 3), or an equivalent of each thereof and the VLP is a QB or a TMV VLP or a mutant or equivalent of each thereof.

In another aspect, the biomimetic ApoAI peptide is chemically conjugated to the outer surface of the nanoparticle by a maleimide-thiol reaction. In some aspects the nanoparticle, VLP or VNP is conjugated to 100-500 peptides per nanoparticle. In other aspects, the nanoparticle, VLP or VLP is conjugated to 400-600 peptides per nanoparticle. The peptides and/or VNP and/or VLP can be the same or different from each other.

In another aspect, the biomimetic ApoAI peptides include a CGGG or CSGGG linker on either the N- or the C-terminus or both.

In one aspect, a plurality of conjugates as described herein are administered wherein the nanoparticles in the plurality can be the same or different from each.

In a further aspect, the conjugates or precursors are detectably labeled.

Modes of administration are known in the art and described herein. Treatment includes restorative as well as preventative therapy. In one aspect, the treatment is restorative therapy only.

The methods can further comprise an administration of another therapy for the treatment of the underlying disorder, e.g., administration of a statin.

The term “subject” includes mammals and human patients. In one aspect, the human patient has one or more of the following conditions: atherosclerosis, cardiovascular disease, or unsafe LDL cholesterol levels.

### Dosage and Dosage Formulations

In some embodiments, the conjugates or compositions are administered to a subject suffering from a condition as disclosed herein, such as a human, either alone or as part of a pharmaceutically acceptable formulation, once a week, once a day, twice a day, three times a day, or four times a day, or even more frequently.

Administration of the conjugates or compositions alone or in combination with an optional additional therapeutic agent and compositions containing same can be accomplished by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

The conjugates or compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

In some embodiments, any of the conjugates or compositions disclosed herein, are administered to the subject at least 1, 2, 3, 4, 5, 6, 7, 8, 9, or 10 times a day. In some embodiments, any of conjugates or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the conjugates or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the conjugates or compositions disclosed herein are administered to the subject at least every 1, 2, 3, 4, 5, 6, 7, 8, 9, or 10 days. In some embodiments, any of the conjugates or compositions disclosed herein are administered to the subject at least every 1, 2, 3, 4, 5, 6, 7, 8, 9, 10, 11, 12, 13, or 14 weeks. In some embodiments, any of the conjugates or compositions disclosed herein are administered to the subject for a period of at least 1, 2, 3, 4, 5, 6, 7, 8, 9, or 10 days. In some embodiments, any of the conjugates or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the conjugates or compositions disclosed herein are administered to the subject for a period of at least 1, 2, 3, 4, 5, 6, 7, 8, 9, 10, 12, 13, 14, 15, 16, 17, 18, 19, or 20 months.

In one aspect, the methods or compositions further comprise administration of an additional therapeutic agent.

In some cases, the additional therapeutic agent comprises, or consists essentially of, or yet further consists of, a salvage therapy.

In some cases, the additional therapeutic agent comprises, or consists essentially of, or yet further consists of, a palliative therapy.

### Diagnostic Methods

In some embodiments, one or more of the methods described herein further comprises, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether the condition has been treated. Assessment of one or more biomarkers associated with a disease or condition, or for characterizing whether a response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

### Kits

In one aspect, the present disclosure provides kits for performing the methods of this disclosure as well as instructions for carrying out the methods of the present disclosure. The kit comprises, or alternatively consists essentially of, or yet further consists of one or more of the multivalent nanoparticle-peptide conjugates, or the plurality of the composition, of this disclosure and instructions for use. In a further aspect, the instruction for use provides directions to conduct any of the methods disclosed herein.

The kit components, (e.g., reagents) can be packaged in a suitable container. The kit can also comprise, or alternatively consist essentially of, or yet further consist of, e.g., a buffering agent, a preservative or a protein-stabilizing agent. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### EXPERIMENTAL

### Materials and Methods

Production of TMV and Qβ. TMV-Lys was propagated in Nicotiana benthamiana as previously described and stored in 0.01 M potassium phosphate (KP) buffer (pH 7.4) at 4° C.^{31,37} TMV-Lys is described as TMV for brevity. Bacteriophage Qβ VLPs were expressed as previously reported and stored in 1×PBS.³⁸

Peptide library. Peptide sequences in Table 1 were selected according to preclinical efficacy in vivo as well as phase I clinical safety and were prepared by solid-phase synthesis (GenScript). The 4F peptide was synthesized with a CSGGG linker on either the N-terminus (4FN) or the C-terminus (4FC) whereas the 23MJ peptide was synthesized without a linker because it contains a native N-terminal cysteine. Sequences were analyzed using Pepcalc (https://pepcalc.com/) to determine characteristics such as molecular weight, isoelectric point, and hydropathicity. Peptide 3D models generated using PEP-FOLD3 were converted to Sybyl mol2 format and imported into ALOGPS 2.1 to calculate LogP values. Peptide sequences and properties are summarized in Table 2.

Bioconjugation of ApoAI peptides to TMV and Qβ external lysine residues. The intermediate particles TMV-(PEG)₄ and Qβ-(PEG)₄ were produced by reacting 2 mg of TMV or Qβ (2 mg mL⁻¹) with SM(PEG)₄ (5 molar equivalents per TMV coat protein and 17 molar equivalents per Qβ coat protein) for 2 h at 25° C. in 0.01 M KP buffer. Particles were purified by ultracentrifugation (50000×g, 1 h, 4° C.) on a 30% (w/v) sucrose cushion in 0.01 M KP buffer. Pellets containing T-M(PEG)₄ and Qβ-M(PEG)₄ were washed twice and resuspended in 0.01 M KP buffer. A maleimide-thiol reaction was then carried out by adding 0.5 molar equivalents of ApoAI peptide (4FC, 4FN or 23MJ) per TMV coat protein or 2.8 molar equivalents per Qβ.

Bioconjugation of 5-Carboxyfluorescein (5FAM) to TMV's internal glutamic acid residues. Intermediate TMV-N₃ particles were produced through a modified protocol.³⁹ Conjugation of interior glutamic acid residues was performed by conjugation with propargylamine (25 molar equivalence per coat protein) and 1-ethyl-3-(3-dimethylaminopropyl)carbodiimide at a final concentration of 2 mg/mL TMV in 0.01 M KP buffer overnight at room temperature. Then, the intermediate was reacted via copper(I)-catalyzed azide-alkyne cycloaddition to 5FAM (two molar equivalents per TMV coat protein) for 30 minutes on ice in 0.01 M KP buffer containing 2 mM aminoguanidine, 2 mM ascorbic acid sodium salt, and 1 mM copper sulfate. Particles were purified by ultracentrifugation (50000×g, 1 h, 4° C.) on a 30% (w/v) sucrose cushion in 0.01 M KP buffer. Pellets containing TMV-5FAM were washed twice and resuspended in 0.01 M KP buffer, then dialyzed overnight against a 0.01 M KP buffer using a Spectrum Labs 10 kDa dialysis membrane (Thermo Fisher Scientific). Purified TMV-5FAM was externally-conjugated to 4FN peptide as described above.

UV/Vis spectroscopy and BCA assay. The concentration of VLPs/VNPs in 0.01 M KP buffer was determined using a NanoDrop spectrophotometer (Thermo Fisher Scientific). Fluorophore modification and the concentration of TMV were determined using the Beer-Lambert law. The extinction coefficient of TMV is F(260 nm)=3 mL·mg⁻¹ cm⁻¹, molecular weight=3.94×10⁷ g mol⁻¹. The extinction coefficient of 5FAM is F(493 nm)=83,000 M⁻¹ cm¹, molecular weight=376 Da. The Qβ concentration was determined by measuring the total protein using a Pierce BCA assay kit (Thermo Fisher Scientific).

Dynamic Light Scattering (DLS). The hydrodynamic diameter of VLPs/VNPs conjugates were probed using a Zetasizer Nano ZSP/Zen5600 instrument (Malvern Panalytical, Malvern, UK). Particle hydrodynamic diameter was calculated as the weighted mean of the intensity distribution and samples were analyzed at 1 mg/mL concentration in 0.01 M KP buffer.

Transmission electron microscopy (TEM). The VLPs/VNPs (0.1 mg/mL) in 10-μL drops of Milli-Q water (0.5 mg mL⁻¹) were added to Formvar FCF400-CU carbon-coated copper TEM grids (Electron Microscopy Sciences) for 2 min at room temperature. The grids were washed twice for 30 s using Milli-Q water and then stained with 10 μL 2% (w/v) uranyl acetate for 1 mi. The samples were analyzed at 80 kV using an FEI Tecnai Spirit G2 Bio TWIN electron microscope.

SDS-PAGE and microfluidic gel electrophoresis. VLP/VNP samples (5 g in 20 L reducing loading dye) were denatured at 100° C. for 5 min and loaded alongside SeeBlue Plus2 marker onto 4-12% or 12% NuPAGE precast gels in 3-(N-morpholino)propanesulfonic acid (MOPS) buffer. After fractionation for 37 min at 200 V and 120 mA, the gels were photographed using the FluorChem R imaging system under white light for Coomassie Brilliant Blue detection and MultiFluor Green light for 5FAM detection. The degree of modification was determined by densitometric band analysis and the calculation of peak integration ratios using ImageJ. Coat proteins and their degree of modification was also analyzed using an Agilent Bioanalyzer 2100 and Protein 80 Kit.

BODIPY-Cholesterol Binding assay. A fluorescent cholesterol, BODIPY-cholesterol or 23-(dipyrrometheneboron difluoride)-24-norcholesterol was used to examine the HDL biomimics' ability to uptake cholesterol. VNP samples (500 μg) and equimolar HDL were incubated with 20 uM BODIPY-cholesterol for 2 hours at 37° C. on a shaker at 150 rpm in 0.5 mL of 10 mM KP/DMSO. After incubation, samples were purified by centrifuge using 100 kDa molecular-weight cutoff Amicon spin-filters to remove free, unbound cholesterol. Samples were analyzed for size on DLS (see above) and fluorescence before and after BODIPY-cholesterol binding using 480/508 nm excitation/emission parameters on a Tecan Plate Reader.

J774 Cholesterol efflux assay. Cholesterol efflux was measured from J774 cells (ATCC) as previously described.²³ Briefly, J774 cells were seeded in 96-well plates at 1×10⁵ cells/100 μL in DMEM (Corning) with 10% (v/v) fetal bovine serum (FBS) and 1% (w/v) penicillin-streptomycine (P/S). Cells were incubated until they reached ˜80% confluence. After two rinses with FBS-free DMEM containing 1% (w/v) P/S they were incubated for 48 h in 200 μL FBS-free DMEM containing 40 μg/mL acetylated low-density lipoprotein (AcLDL), 2 μg/mL Sandoz inhibitor, 1% (w/v) P/S, and 5% (v/v) human lipoprotein-deficient serum (LPDS) to produce foam cells. Excess lipids were removed by two rinses as above, and foam cells were equilibrated for 12 h in 200 μL FBS-free DMEM, 2 μg/mL Sandoz inhibitor, 0.3 mM CPT-cAMP, 1% (w/v) P/S, and 5% (v/v) human LPDS. After two further rinses, cholesterol efflux was initiated by adding different VLP/VNP or peptide treatments in FBS-free DMEM containing 1% (w/v) P/S and 5% (v/v) human LPDS and incubating for 15 h. The medium was then collected, centrifuged (1000×g, 10 min, 4° C.) and the cholesterol content of the supernatant was determined using the Amplex assay kit. Cells were washed three times with ice-cold PBS, lysed using 200 μL of Cytobuster lysis buffer, and the cholesterol content of the lysate was determined using the Amplex assay kit. Cholesterol efflux was calculated by dividing the amount of cholesterol in the medium by the total cholesterol content of the medium and lysate.

ROS assay. SVEC cells (ATCC) were seeded in four-compartment 35-mm CELLview glass-bottom dishes or 96-well plates at 6×10⁴ cells/mL in 500 μL DMEM and were allowed to reach ˜80% confluence. The medium was then removed, and the cells were washed once with PBS before incubation with VLPs/VNPs for 8 h. Applicant then added 100 μg/mL ox-LDL for 4 h to induce oxidative stress and detected ROS using a CellROX Green Reagent Kit. Images were captured using a Nikon AiR confocal microscope with a 20×, 0.75 numerical aperture dry objective.

Wound healing assay. SVEC cell migration was investigated using a Radius 96-well kit (Cell Bio Labs). Applicant seeded 3×10⁴ cells in 200 μL DMEM per well and allowed the cells to reach ˜80% confluence. After removing the Radius Gel, cells were allowed to migrate for 24 h in the presence of various treatments. Bright-field phase contrast images were acquired after 0, 12 and 24 h using an EVOS FL microscope fitted with a 10× objective (n=6 cells per group). Percentage wound closure was calculated by tracing the wound perimeter in ImageJ to calculate the area at t=12 or 24 h and dividing by the initial starting area at t=0 h.

TMV-ApoAI distribution during cholesterol efflux from J774 foam cells. Applicant seeded 1×10⁵ J774 cells in 500 μL DMEM containing 10% (v/v) FBS and 1% (w/v) P/S into each zone of a four-compartment 35-mm CELLview glass-bottom dish, and allowed them to reach ˜80% confluence. Cells were rinsed twice with 500 μL FBS-free DMEM containing 1% (w/v) P/S and incubated for 48 h in 200 μL FBS-free DMEM containing 40 μg/mL of fluorescence-labeled acetylated low-density lipoprotein (Dil-AcLDL), 2 μg/mL Sandoz inhibitor, 1% (w/v) P/S, and 5% (v/v) human LPDS to produce labeled foam cells. After two rinses as above, foam cells were equilibrated for 12 h in 200 μL FBS-free DMEM containing 2 μg/mL Sandoz inhibitor, 0.3 mM CPT-cAMP, 1% (w/v) P/S, and 5% (v/v) human LPDS. After two further rinses, the cells were treated with 100 μL of 5FAM-labeled treatments and controls in FBS-free DMEM containing 1% (w/v) P/S and 5% (v/v) human LPDS. After 12 h, the cells were washed twice with 500 μL PBS and then fixed for 10 minutes at room temperature using a PBS solution containing 10% (w/v) PFA, 25% (w/v) glutaraldehyde. Cells were then twice washed with PBS and subsequently stained for 45 min with 300 μL of wheat-germ agglutinin AlexaFluor 647 (WGA-647) prepared in PBS and 5% (v/v) goat serum at a 1:500 dilution. Cells were then twice washed with PBS and then stained with 300 μL DAPI dye (5 μg/mL in PBS) for 20 min. Cells were washed twice with PBS and then imaged using a Nikon AiR confocal microscope with a 20×, 0.75 numerical aperture dry objective.

Statistical methods. Samples were prepared in triplicate unless otherwise stated. Data were processed by one-way analysis of variance (ANOVA) with Tukey's test (*p<0.05, **p<0.01, ***p<0.001, ****p<0.0001, ns=not significant) using Graphpad Prism 6.

### Experimental Discussion

Applicant evaluated two viral nanocarriers differing in shape and size. First, bacteriophage Qβ (Qβ) is a 30-nm spherical virus-like particle (VLP) with 180 identical coat protein units arranged in T3 symmetry, allowing peptide conjugation to amine groups on the surface. Second, tobacco mosaic virus (TMV) is a rod-shaped plant virus nanoparticle (VNP) with a high aspect ratio (300×18 nm) composed of 2130 coat protein units that self-assemble around a single plus-sense single-stranded RNA (Table 1). Applicant used a TMV mutant with lysine in place of alanine at position 158 (TMV-Lys) to facilitate conjugation.³¹ Applicant evaluated three biomimetic ApoAI peptides: 4FN and 4FC (based on the clinical 4F sequence with a cysteine added via intervening GGGS-linker for conjugation at the N- or C terminus) as well as 23MJ (based on a preclinical multivalent formulation) (Table 2).

Applicant evaluated the mechanistic and functional quality of TMV-ApoAI conjugates in cholesterol binding assays, cholesterol efflux assays, reactive oxygen species (ROS) assays, and wound healing assays.

TABLE 1
Model nanoparticles.

| | | | Number of | Number of | |
|---|---|---|---|---|---|
| | | Size | coat | surface | |
| VNP/VLP | Shape | (nm) | proteins | lysines | Ref. |
| TMV-Lys | High-aspect | 300 × 30 | 2130 | 2130 | ²⁹ |
| (TMV) | ratio, rigid rod | | | | |
| Qβ | Icosahedral | 28 | 180 | 720 | ³⁵ |

TABLE 2
Biomimetic ApoAI peptides.

| | | | | Hydrophobicity <H> | |
|---|---|---|---|---|---|
| Peptide | | MW | | (hydrophobic moment) | |
| ID | Sequence (length) | (kDa) | LogP | <μH> | Ref. |
| 4FN | CSGGGDWFKAFYDKVAEKFKEAF | ~2.63 | −1.83 | 0.315 (0.510) | 38 |
| | (23) | | (0.315) | | |
| 4FC | DWFKAFYDKVAEKFKEAFGGGSC | ~2.63 | −1.86 | 0.315 (0.393) | 38 |
| | (23) | | (0.315) | | |
| 23MJ | CGVLESFKASFLSALEEWTKKLQ | ~2.61 | −2.59 | 0.480 (0.508) | 22 |
| | (23) | | (0.480) | | |

### Results and Discussion

As noted herein, the term “TMV” as used in the Material and Methods, and Experimental Discussion, intends the TMV-lys mutant, identified above.

To investigate the efficacy of multivalent biomimetic ApoAI peptides displayed on the surface of viruses, Applicant combined three model peptides (Table 1) with two carriers: a bacteriophage derived VLP and a VNP based on a plant virus, the former serving as a model for a nanoparticle and the latter as nanotube (Table 2). All three biomimetic ApoAI peptides are derived from functional ca-helix regions (FIG. 1) and have proven to be safe during clinical trials (4F) or efficacious in terms of cholesterol efflux during preclinical development (23MJ). The 4F peptide was synthesized with a CSGGG linker to attach either the N-terminus (4FN) or C-terminus (4FC) to the carrier, allowing us to investigate the implications of peptide orientation, whereas the 23MJ peptide contained a native N-terminal cysteine which removed the need for a linker (FIG. 1A).

Peptide conjugates (FIG. 1B) were synthesized in a two-part reaction. First, heterobifunctional linker SM(PEG)₄ was reacted with the VLPs/VNPs to form the maleimide intermediates T-M(PEG)₄ and Qβ-M(PEG)₄. Second, each intermediate was then reacted with the three ApoAI peptides and the final particles termed TMV-ApoAI and Qβ-ApoAI were purified by ultracentrifugation.

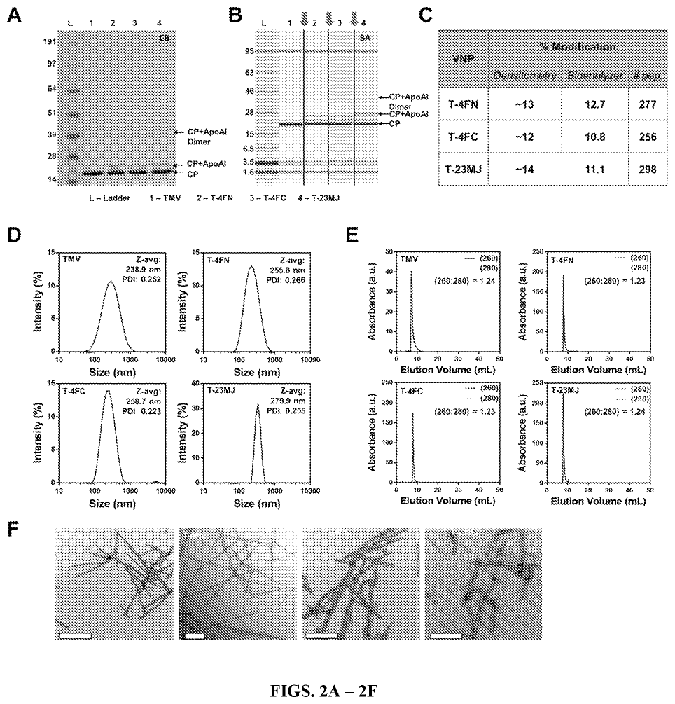

The conjugation of ApoAI peptides to TMV was confirmed by SDS-PAGE, which revealed the presence of ˜20-kDa bands in addition to ˜17.5-kDa bands corresponding to conjugated and unmodified TMV coat protein, respectively (FIG. 2A). The degree of modification was ˜12-14% as determined by densitometric analysis, agreeing with the ˜11-13% modification as determined using the bioanalyzer (FIG. 2B). These values represent ˜250-300 peptides per TMV particle (FIG. 2C). Dynamic light scattering (DLS) indicated a size increase for the modified TMV-ApoAI particles: TMV had a Zₐᵥ of ˜203.1 nm (PDI=0.230), a ˜1.3-fold size increase was observed for the ApoAI-conjugates with: Zₐᵥ˜255.8 nm (PDI=0.266) for T-4FN, Zₐᵥ˜258.7 nm (PDI=0.223) for TMV-4FC and Zₐᵥ˜279.9 nm (PDI=0.255) for TMV-23MJ (FIG. 2D). FPLC data indicate that TMV-ApoAI peptide conjugates exhibited typical elution profile of TMV at retention volume of ˜7.5 mL along with a 260:280 nm absorbance ratio of ˜1.2 (FIG. 2E). Additionally, the analysis of negatively stained TMV particles by TEM further confirm that the particle morphology remained intact after modification (FIG. 2F). Together, these results confirm the bioconjugation of ApoAI peptides to TMV yielding intact TMV-ApoAI formulations.

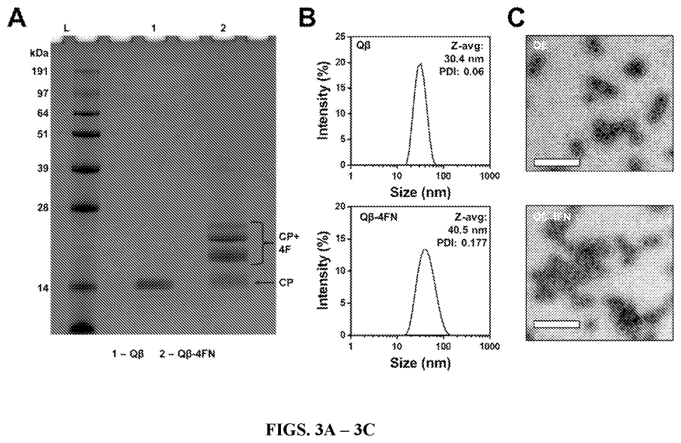

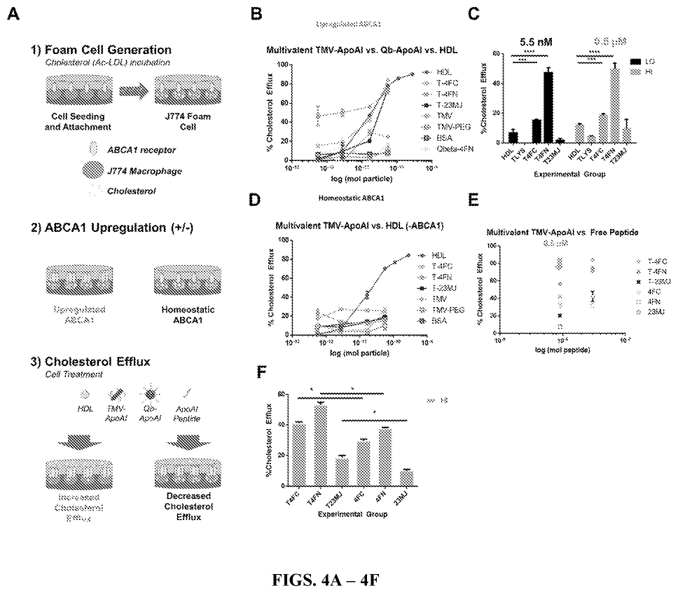

SDS-PAGE also confirmed the conjugation of Qβ to ApoAI peptide 4FN, revealing the presence of unmodified CP bands at ˜14.2 kDa plus multiple modified CP bands with higher molecular weights (FIG. 3A). The degree of modification was ˜55-60% as determined by densitometric analysis. DLS indicated that the hydrodynamic diameter of Qβ-4FN was slightly larger (Zₐᵥ˜40.5 nm, PDI=0.177) than that of unmodified Qβ (Zₐᵥ˜30.4 nm, PDI=0.06) (FIG. 3B). The structural integrity of the particles was confirmed by TEM (FIG. 3C). Applicant note that Qβ was modified only with 4FN; initial scouting experiments were performed with TMV and the three peptide candidates which revealed that 4FN was the most potent peptide (see below). Therefore, questions about the impact of the nanoparticle size/shape were only addressed using T-4FN v. Qβ-4FN. Synthesized TMV-ApoAI and Qβ-ApoAI conjugates were then employed in a cholesterol efflux assay (FIG. 4A).

Data indicate T-4FN is a more potent cholesterol binder than TMV-4FC (˜2.5-fold), which suggests that cholesterol efflux using multivalent display of 4F peptide is highly orientational-dependent (FIGS. 4B-4C). In addition, spherical Qβ-ApoAI peptide conjugate does not display significant cholesterol efflux and is comparable to baseline BSA control group efflux. Therefore, rod-shaped nanocarrier is a more suitable candidate for further investigation of multivalent peptide display effect. Structural work indicates that both the N and C termini of ApoAI peptide were involved in lipid and cholesterol binding. However, Applicant's findings demonstrate that potent efflux can still be observed in an ABCA1-dependent manner with the N-terminus of the peptide reserved for conjugation (FIG. 4B and FIG. 4D). Moreover, the terminus-specific efflux observed in Applicant's study suggest a more complex cholesterol binding structure is at play, given that when ABCA1 receptor is not upregulated, the observed cholesterol efflux of T-4FN drastically decreases (FIG. 4D). Further structural work should be conducted on nanoparticle-ApoAI peptide conjugates to identify potential supramolecular assemblies afforded by surface engineering of ApoAI peptides that significantly boost the cholesterol efflux compared to equimolar free peptide doses (FIGS. 4E and 4F). Of note, non-covalent lipid nanoparticle formulations have been the mainstay to ApoAI peptide delivery, but Applicant's findings suggest nanocarrier-conjugated delivery is a viable, potentially more potent approach.

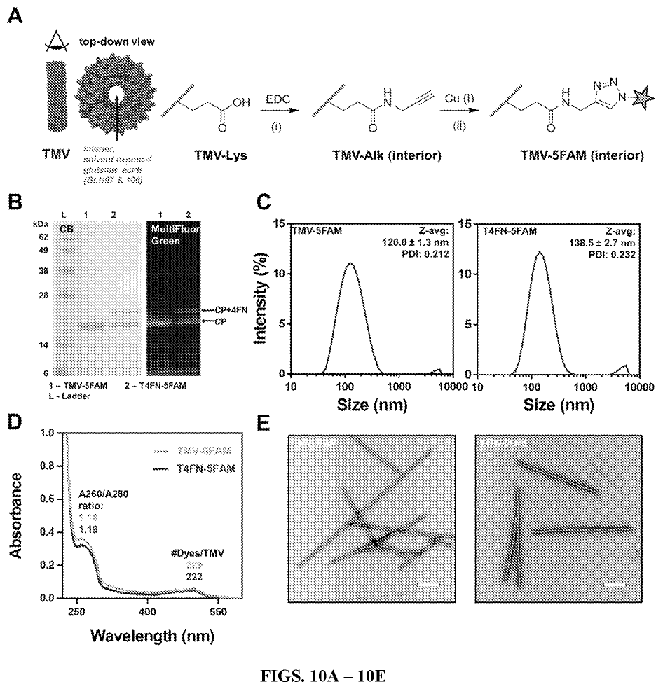

Applicant's in vitro functional assays confirmed that the T-4FN candidate shows promise in the context of cardiovascular therapy. To investigate the underlying molecular mechanism, Applicant generated a fluorescence-labeled T-4FN construct for 3D confocal scanning to visualize the cellular localization of the VNPs during cholesterol efflux. This was achieved by converting internal glutamic acid residues lining the TMV inner channel (FIG. 10) to alkyne groups by EDC-mediated conjugation with propargylamine, followed by the copper(I)-catalyzed azide-alkyne cycloaddition of 5FAM. The TMV-5FAM intermediate was then externally conjugated with the 4FN peptide using maleimide-thiol chemistry via an SM(PEG)₄ intermediate as discussed above, producing the final fluorescent conjugate T-4FN-5FAM.

Following purification, the conjugation of TMV-5FAM to 4FN peptides was confirmed by SDS-PAGE, which revealed the presence of conjugate coat protein bands at ˜20 kDa and unmodified coat protein bands at ˜17.5 kDa. Densitometric analysis indicated a ˜20% degree of modification, and the presence of 5FAM on both the modified and unmodified coat proteins was confirmed by the MultiFluor Green signal (FIG. 10B). DLS indicated that the T-4FN-5FAM conjugate (Zₐᵥ˜138.5 nm, PDI=0.232) was slightly larger than TMV-5FAM (Zₐᵥ˜120.0 nm, PDI=0.212) (FIG. 10C). UV/Vis spectrophotometry indicated that ˜220-230 5FAM molecules were present per TMV particle. A260/A280 ratios were around the expected value of ˜1.2 (FIG. 10D). TEM confirmed the presence of intact T-4FN-5FAM particles (FIG. 10E).

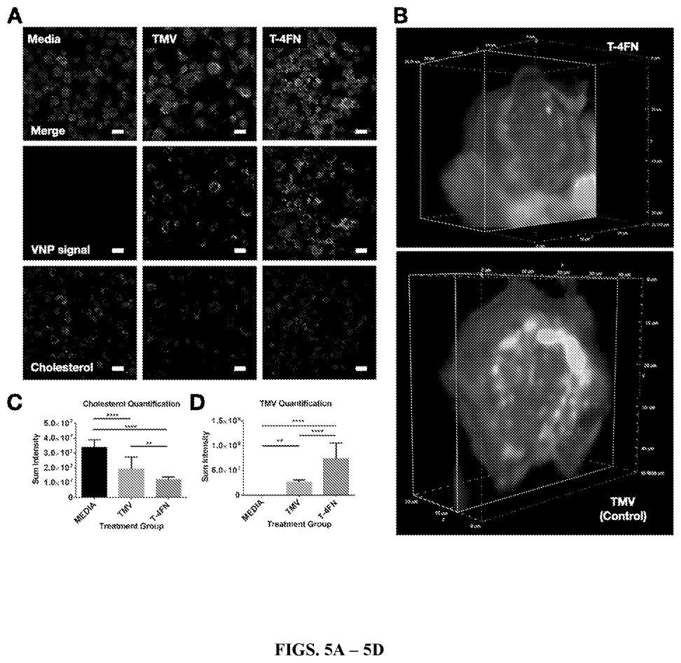

A fluorescently labeled T-4FN-5FAM was used to promote cholesterol efflux from fluorescently-labeled foam cells (FIG. 5A and FIG. 15B). Cholesterol and T-4FN-5FAM fluorescence was quantified using Nikon AR Elements Analysis software's 3D volume viewer tool and plotted in a bar graph (FIG. 5C and FIG. 5D).

The localization of T-4FN particles differed strikingly from that of unmodified TMV. Punctate foci containing TMV particles were mostly observed within the foam cells whereas most of the T-4FN particles were extracellular, forming a dense layer on the plasma membrane (FIG. 5A and FIG. 5B). It was evident that the formed layer was tightly associated due to its persistent membrane attachment following repeated PBS washes prior to fixing. Dil-LDL cholesterol fluorescence intensity was significantly lower for the T-4FN treatment compared to unmodified TMV (p<0.01) (FIG. 5C), consistent with the cholesterol efflux assay using non-fluorescent particle and cholesterol constituents. Interestingly, TMV itself promotes a baseline level of cholesterol efflux, augmented by the multivalent display of 4FN. The fluorescence intensity of 5FAM was significantly higher for T-4FN particles than unmodified TMV (p<0.0001). There was also an inverse correlation of T-4FN cell binding and cholesterol efflux (FIG. 5C and FIG. 5D).

To investigate the ability of the TMV particles to bind cholesterol, Applicant mixed them with fluorescent BODIPY cholesterol for 2 h at 37° C. in 0.01M KP/DMSO (50:50).

The cholesterol-bound nanoparticles were separated from free cholesterol by centrifugation in spin filters with a 100-kDa cutoff. Samples without cholesterol were incubated as a negative control. Purified fractions were analyzed by DLS and fluorimetry.

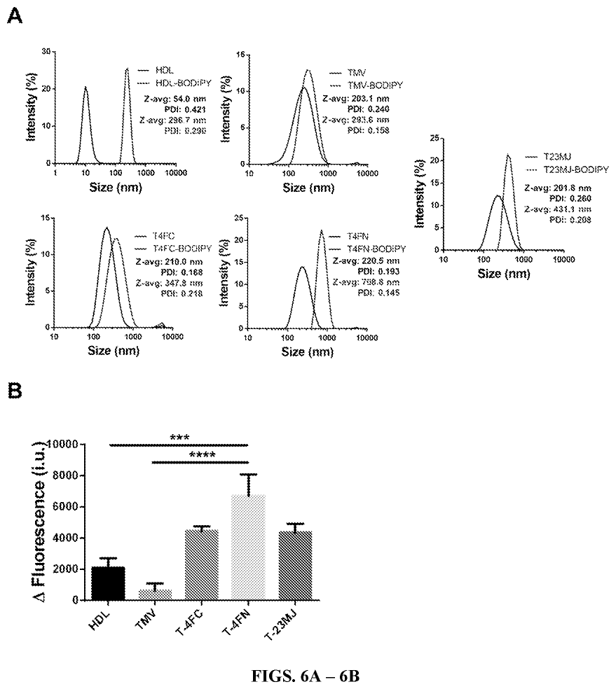

Applicant observed an increase in the size of all cholesterol-incubated samples relative to cholesterol-free controls. The Zₐᵥ of the HDL positive control increased from 54.0 to 296.7 nm, while the PDI decreased from 0.421 to 0.290. However, the greatest change was observed for the T-4FN conjugate, where the Zₐᵥ increased from 220.5 to 708.8 nm, while the PDI decreased from 0.193 to 0.145 (FIG. 6A). Interestingly, unmodified TMV showed an inherent affinity for cholesterol, as revealed by the Zₐᵥ increase from 2o3.1 to 293.6 nm, while the PDI decreased from 0.240 to 0.158. The lower PDI of most samples following exposure to cholesterol suggests that cholesterol binding improves order among particle populations, although TMV-4FC was an exception, showing an increase in PDI from 0.168 to 0.218 (FIG. 6A). The change in fluorescence was calculated by subtracting the signal before cholesterol binding from the signal after cholesterol binding. T-4FN produced the most intense signal, consistent with the size increase observed by DLS (FIG. 6B).

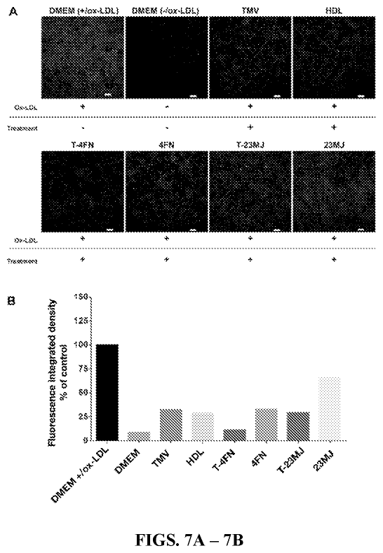

Given the encouraging cholesterol efflux data confirming ABCA1-mediated cholesterol efflux as well as free cholesterol binding mechanisms of Applicant's biomimetic multivalent ApoAI peptide nanoparticles, Applicant carried out additional functional assays to investigate potential orthogonal benefits of Applicant's conjugates beyond cholesterol removal. HDL and the 4F peptide are known to activate endothelial nitric oxide synthase (eNOS) and upregulate nitric oxide (NO), which has atheroprotective properties. Chronic oxidative stress and inflammation are major factors driving endothelial cell dysfunction, which promotes the accumulation of oxidized-LDL in the lumen, foam cell generation, and atherosclerosis. Using an SVEC endothelial cell line, which was subject to treatment and oxidative stress periods, Applicant assessed the ability of Applicant's multivalent TMV-ApoAI to mitigate the formation of ROS (FIG. 7).

The cells were treated with 0.5 μM HDL, 0.5 μM TMV, and varying amounts of free peptide normalized depending to the TMV-ApoAI conjugates (particle treatment groups were matched on a 1:1 molar equivalent basis with HDL, whereas peptide treatment groups were matched on a 1:1 molar equivalent basis with the number of peptides displayed per TMV particle (FIG. 7A). After treatment, Applicant induced oxidative stress by exposing the cells to 50 μg/ml ox-LDL. Applicant found that all treatment groups were able to mitigate ROS production, with T-4FN reducing the ROS signal to near the level of the negative media control, lacking ox-LDL (FIG. 7B). 23MJ peptide did not mitigate ROS, however, this could be due to the fact that it is a class Y alpha helix compared to class A alpha helix; class Y preferentially associates with lipids over LDL-cholesterol, which in the ROS assay was the primary driver of inflammation.

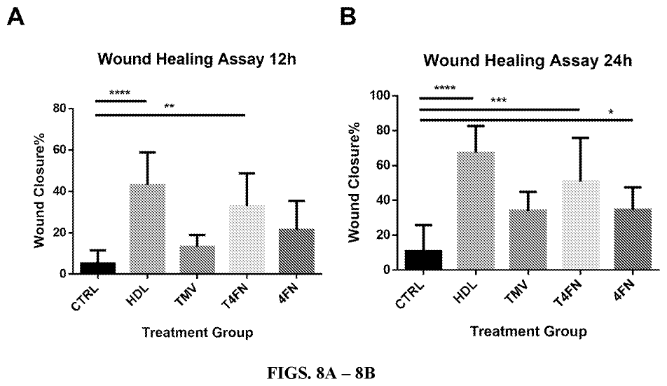

The cholesterol efflux and ROS assay data suggested that T-4FN was the most promising candidate for further functional investigation. Applicant therefore evaluated the effect of T-4FN on wound healing using SVEC cells. To better recapitulate the wound healing environment during in endothelial cell dysfunction, Applicant combined a traditional scratch wound healing assay with cellular oxidative stress.⁴⁰ Applicant compared the effects of 8-h treatments with 0.5 μM HDL, 4FN, T-4FN or unmodified TMV followed by 4-h exposure to 50 μg/mL ox-LDL on wound closure for 12 and 24 h following 8-h treatments (FIGS. 8A and 8B).

Exposing the SVEC cells to HDL enhanced wound closure after 12 and 24 h compared with the control (serum-free medium). After 12 h, 0.5 μM HDL and 0.5 μM T-4FN increased wound closure by 8.42-fold (p<0.0001) and 6.41-fold (p<0.01), respectively (FIG. 8A). After 24 h, 0.5 μM HDL and 0.5 μM T-4FN increased wound closure by 6.24-fold (p<0.0001) and 4.70-fold (p<0.001), respectively, and even the free 4FN peptide increased wound closure by 3.20-fold (p<0.05) (FIG. 8B). Unmodified TMV also improved wound closure by 2.58-fold after 12 h and 3.15-fold after 24 h. Although the difference is not statistically significant, T-4FN performed better than the 4FN peptide at both time points, suggesting that multivalent display can enhance endothelial cell wound healing.

### Experimental Discussion

Applicant has demonstrated for the first time that the multivalent display of ApoAI peptides on plant-based VNPs enhances cholesterol efflux, ROS mitigation, and wound healing compared to HDL or free ApoAI peptides. Applicant also found that ABCA1-mediated efflux can occur without both peptide termini free. Applicant report several critical design principles regarding VNP/VLP-ApoAI peptide conjugates, which may apply to other nanoparticle systems: (1) ABCA1-mediated cholesterol efflux is terminus-dependent (T-4FN vs TMV-4FC), (2) ABCA1-mediated cholesterol efflux is influenced by the nanoparticle shape, being more effective with TMV rods than Qβ particles, and (3) multivalent ApoAI display changes the foam cell localization of nanoparticles. Importantly, the clinical safety of several ApoAI peptides in addition to 4F has been validated, and these may also benefit from multivalent display on VNPs. Unmodified TMV also showed unanticipated inherent cholesterol binding and ROS-mitigating properties, which should be further investigated, possibly using a library of plant viruses. The analysis of novel combinations of nanoparticles and ApoAI peptides may reinvigorate the cholesterol efflux approach to combat atherosclerosis in vivo.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Partial Sequence Listing
SEQ ID 1:
CSGGGDWFKAFYDKVAEKFKEAF(4FN)
SEQ ID 2:
DWFKAFYDKVAEKFKEAFGGGSC(4FC)
SEQ ID 3:
CGVLESFKASFLSALEEWTKKLQ(23MJ)
SEQ ID 4:
MSYSITTPSQFVFLSSAWADPIELINLCTNALGNQFQTQQARTVVQRQF
SEVWKPSPQVTVRFPDSDFKVYRYNAVLDPLVTALLGAFDTRNRIIEVE
NQANPTTAETLDATRRVDDATVAIRSAINNLIVELIRGTGSYNRSSFES
SSGLVWTSGPAT(TMV)
SEQ ID 5:
MSYSITTPSQFVFLSSAWADPIELINLCTNALGNQFQTQQARTVVQRQF
SEVWKPSPQVTVRFPDSDFKVYRYNAVLDPLVTALLGAFDTRNRIIEVE
NQANPTTAETLDATRRVDDATVAIRSAINNLIVELIRGTGSYNRSSFES
SSGLVWTSGPK(TMV-LYS)
SEQ ID 6:
MAKLETVTLGNIGKDGKQTLVLNPRGVNPTNGVASLSQAGAVPALEKRV
TVSVSQPSRNRKNYKVQVKIQNPTACTANGSCDPSVTRQAYADVTFSFT
QYSTDEERAFVRTELAALLASPLLIDAIDQLNPAY(QB)

### REFERENCES

(1) Lusis, A. J. Atherosclerosis. Nature 2000, 407 (6801), 233-241. https://doi.org/10.1038/35025203.
(2) Libby, P.; Buring, J. E.; Badimon, L.; Hansson, G. K.; Deanfield, J.; Bittencourt, M. S.; Tokgozoglu, L.; Lewis, E. F. Atherosclerosis. Nat Rev Dis Primers 2019, 5 (1), 1-18. https://doi.org/10.1038/s41572-019-0106-z.
(3) Heart Disease and Stroke Statistics 2021 Update | Circulation. https://www.ahajournals.org/doi/10.1161/CIR.0000000000000950 (accessed 2022-06-22).
(4) Dagenais, G. R.; Leong, D. P.; Rangarajan, S.; Lanas, F.; Lopez-Jaramillo, P.; Gupta, R.; Diaz, R.; Avezum, A.; Oliveira, G. B. F.; Wielgosz, A., et al., Variations in Common Diseases, Hospital Admissions, and Deaths in Middle-Aged Adults in 21 Countries from Five Continents (PURE): A Prospective Cohort Study. The Lancet 2020, 395 (10226), 785-794. https://doi.org/10.1016/S0140-6736(19)32007-0.
(5) Weber, C.; Noels, H. Atherosclerosis: Current Pathogenesis and Therapeutic Options. Nat Med 2011, 17 (11), 1410-1422. https://doi.org/10.1038/nm.2538.
(6) Immunity and Inflammation in Atherosclerosis. https://www.ahajournals.org/doi/epub/10.1161/CIRCRESAHA.118.313591 (accessed 2022-06-22). https://doi.org/10.1161/CIRCRESAHA.118.313591.
(7) Soehnlein, O.; Libby, P. Targeting Inflammation in Atherosclerosis from Experimental Insights to the Clinic. Nat Rev Drug Discov 2021, 20 (8), 589-610. https://doi.org/10.1038/s41573-021-00198-1.
(8) Kingwell, B. A.; Chapman, M. J. Future of High-Density Lipoprotein Infusion Therapies. Circulation 2013, 128 (10), 1112-1121. https://doi.org/10.1161/CIRCULATIONAHA.113.002683.
(9) Huang, Y.; DiDonato, J. A.; Levison, B. S.; Schmitt, D.; Li, L.; Wu, Y.; Buffa, J.; Kim, T.; Gerstenecker, G.; et al., An Abundant Dysfunctional Apolipoprotein A1 in Human Atheroma. Nat Med 2014, 20 (2), 193-203. https://doi.org/10.1038/nm.3459.
(10) Reiner, Ž. Resistance and Intolerance to Statins. Nutrition, Metabolism and Cardiovascular Diseases 2014, 24 (10), 1057-1066. https://doi.org/10.1016/j.numecd.2014.05.009.
(11) Sinning, D.; Landmesser, U. Low-Density Lipoprotein-Cholesterol Lowering Strategies for Prevention of Atherosclerotic Cardiovascular Disease: Focus on SiRNA Treatment Targeting PCSK9 (Inclisiran). Curr Cardiol Rep 2020, 22 (12), 176. https://doi.org/10.1007/si 1886-020-01427-6.
(12) Novartis receives EU approvalfor Leqvio@* (inclisiran), afirst-in-class siRNA to lower cholesterol with two doses a year**. Novartis. https://www.novartis.com/news/media-releases/novartis-receives-eu-approval-leqvio-inclisiran-first-class-sirna-lower-cholesterol-two-doses-year (accessed 2022-06-22).
(13) 7-Lipoprotein and Lipid Metabolism | Elsevier Enhanced Reader. https://reader.elsevier.com/reader/sd/pii/B9780128125359000078?token=3F56BB9140C1B7 776F8C0E3ED9BE06138688D13BB5C69D35B497E74096A23CB29B5755305310566A3E C8F2848871544B&originRegion=us-east-1&originCreation=20220622175649 (accessed 2022-06-22). https://doi.org/10.1016/B978-O-12-812535-9.00007-8.
(14) Takata, K.; Di Bartolo, B. A.; Nicholls, S. J. High-Density Lipoprotein Infusions. Cardiology Clinics 2018, 36(2), 311-315. https://doi.org/10.1016/j.ccl.2017.12.012.
(15) Rohatgi, A.; Khera, A.; Berry, J. D.; Givens, E. G.; Ayers, C. R.; Wedin, K. E.; Neeland, I. J.; Yuhanna, I. S.; Rader, D. R.; et al., HDL Cholesterol Efflux Capacity and Incident Cardiovascular Events. N Engl J Med 2014, 371 (25), 2383-2393. https://doi.org/10.1056/NEJMoa1409065.
(16) He, H.; Hong, K.; Liu, L.; Schwendeman, A. Artificial High-Density Lipoprotein-Mimicking Nanotherapeutics for the Treatment of Cardiovascular Diseases. WIREs Nanomedicine and Nanobiotechnology 2021, 13 (6), e1737. https://doi.org/10.1002/wnan.1737.
(17) Bonizzi, A.; Piuri, G.; Corsi, F.; Cazzola, R.; Mazzucchelli, S. HDL Dysfunctionality: Clinical Relevance of Quality Rather Than Quantity. Biomedicines 2021, 9 (7), 729. https://doi.org/10.3390/biomedicines9070729.
(18) Martinez-López, D.; Camafeita, E.; Cedó, L.; Roldan-Montero, R.; Jorge, I.; Garcia-Marqués, F.; Gómez-Serrano, M.; Bonzon-Kulichenko, E.; Blanco-Vaca, F.; Blanco-Colio, L. M.; et al., APOA1 Oxidation Is Associated to Dysfunctional High-Density Lipoproteins in Human Abdominal Aortic Aneurysm. eBioMedicine 2019, 43, 43-53. https://doi.org/10.1016/j.ebiom.2019.04.012.
(19) Javaheri, A.; Rader, D. J. Apolipoprotein A-I and Cholesterol Efflux. Circulation Research 2014, 114 (11), 1681-1683. https://doi.org/10.1161/CIRCRESAHA.114.303974.
(20) Hamer, M.; O'Donovan, G.; Stamatakis, E. High-Density Lipoprotein Cholesterol and Mortality. Arteriosclerosis, Thrombosis, and Vascular Biology 2018, 38 (3), 669-672. https://doi.org/10.1161/ATVBAHA.117.310587.
(21) Kosmas, C. E.; Martinez, I.; Sourlas, A.; Bouza, K. V.; Campos, F. N.; Torres, V.; Montan, P. D.; Guzman, E. High-Density Lipoprotein (HDL) Functionality and Its Relevance to Atherosclerotic Cardiovascular Disease. DIC 2018, 7, 1-9. https://doi.org/10.7573/dic.212525.
(22) Nissen, S. E.; Tsunoda, T.; Tuzcu, E. M.; Schoenhagen, P.; Cooper, C. J.; Yasin, M.; Eaton, G. M.; Lauer, M. A.; Sheldon, W. S.; Grines, C. L.; et al., Effect of Recombinant ApoA-I Milano on Coronary Atherosclerosis in Patients With Acute Coronary Syndromes: A Randomized Controlled Trial. JAMA 2003, 290 (17), 2292-2300. https://doi.org/10.1001/jama.290.17.2292.
(23) Zhao, Y.; Imura, T.; Leman, L. J.; Curtiss, L. K.; Maryanoff, B. E.; Ghadiri, M. R. Mimicry of High-Density Lipoprotein: Functional Peptide-Lipid Nanoparticles Based on Multivalent Peptide Constructs. J. Am. Chem. Soc. 2013, 135 (36), 13414-13424. https://doi.org/10.1021/ja404714a.
(24) Zhao, Y.; Black, A. S.; Bonnet, D. J.; Maryanoff, B. E.; Curtiss, L. K.; Leman, L. J.; Ghadiri, M. R. In Vivo Efficacy of HDL-like Nanolipid Particles Containing Multivalent Peptide Mimetics of Apolipoprotein A-I[S]. Journal of Lipid Research 2014, 55 (10), 2053-2063. https://doi.org/10.1194/jlr.M049262.
(25) Bloedon, L. T.; Dunbar, R.; Duffy, D.; Pinell-Salles, P.; Norris, R.; DeGroot, B. J.; Movva, R.; Navab, M.; Fogelman, A. M.; Rader, D. J. Safety, Pharmacokinetics, and Pharmacodynamics of Oral ApoA-I Mimetic Peptide D-4F in High-Risk Cardiovascular Patients. Journal of Lipid Research 2008, 49 (6), 1344-1352. https://doi.org/10.1194/jlr.P800003-JLR200.
(26) Dunbar, R. L.; Movva, R.; Bloedon, L. T.; Duffy, D.; Norris, R. B.; Navab, M.; Fogelman, A. M.; Rader, D. J. Oral Apolipoprotein A-I Mimetic D-4F Lowers HDL-Inflammatory Index in High-Risk Patients: A First-in-Human Multiple-Dose, Randomized Controlled Trial. Clinical and Translational Science 2017, 10 (6), 455-469. https://doi.org/10.1111/cts.12487.
(27) White, C. R.; Datta, G.; Wilson, L.; Palgunachari, M. N.; Anantharamaiah, G. M. The ApoA-I Mimetic Peptide 4F Protects Apolipoprotein A-I from Oxidative Damage. Chem Phys Lipids 2019, 219, 28-35. https://doi.org/10.1016/j.chemphyslip.2019.01.009.
(28) Nandwana, V.; Ryoo, S.-R.; Kanthala, S.; McMahon, K. M.; Rink, J. S.; Li, Y.; Venkatraman, S. S.; Thaxton, C. S.; Dravid, V. P. High-Density Lipoprotein-like Magnetic Nanostructures (HDL-MNS): Theranostic Agents for Cardiovascular Disease. Chem. Mater. 2017, 29 (5), 2276-2282. https://doi.org/10.1021/acs.chemmater.6b05357.
(29) Henrich, S. E.; Hong, B. J.; Rink, J. S.; Nguyen, S. T.; Thaxton, C. S. Supramolecular Assembly of High-Density Lipoprotein Mimetic Nanoparticles Using Lipid-Conjugated Core Scaffolds. J. Am. Chem. Soc. 2019, 141 (25), 9753-9757. https://doi.org/10.1021/jacs.9b00651.
(30) Kuai, R.; Li, D.; Chen, Y. E.; Moon, J. J.; Schwendeman, A. High-Density Lipoproteins: Nature's Multifunctional Nanoparticles. ACS Nano 2016, 10 (3), 3015-3041. https://doi.org/10.1021/acsnano.5b07522.
(31) Geiger, F. C.; Eber, F. J.; Eiben, S.; Mueller, A.; Jeske, H.; Spatz, J. P.; Wege, C. TMV Nanorods with Programmed Longitudinal Domains of Differently Addressable Coat Proteins. Nanoscale 2013, 5 (9), 3808-3816. https://doi.org/10.1039/C3NR33724C.
(32) Pitek, A. S.; Wang, Y.; Gulati, S.; Gao, H.; Stewart, P. L.; Simon, D. I.; Steinmetz, N. F. Elongated Plant Virus-Based Nanoparticles for Enhanced Delivery of Thrombolytic Therapies. Mol. Pharmaceutics 2017, 14 (11), 3815-3823. https://doi.org/10.1021/acs.molpharmaceut.7b00559.
(33) Park, J.; Gao, H.; Wang, Y.; Hu, H.; Simon, D. I.; Steinmetz, N. F. S100A9-Targeted Tobacco Mosaic Virus Nanoparticles Exhibit High Specificity toward Atherosclerotic Lesions in ApoE−/− Mice. J. Mater. Chem. B 2019, 7 (11), 1842-1846. https://doi.org/10.1039/C8TB02276C.
(34) Park, J.; Wen, A. M.; Gao, H.; Shin, M. D.; Simon, D. I.; Wang, Y.; Steinmetz, N. F. Designing S100A9-Targeted Plant Virus Nanoparticles to Target Deep Vein Thrombosis. Biomacromolecules 2021, 22 (6), 2582-2594. https://doi.org/10.1021/acs.biomac.1c00303.
(35) Bruckman, M. A.; Jiang, K.; Simpson, E. J.; Randolph, L. N.; Luyt, L. G.; Yu, X.; Steinmetz, N. F. Dual-Modal Magnetic Resonance and Fluorescence Imaging of Atherosclerotic Plaques in Vivo Using VCAM-1 Targeted Tobacco Mosaic Virus. Nano Lett. 2014, 14 (3), 1551-1558. https://doi.org/10.1021/nl404816m.
(36) Wen, A. M.; Wang, Y.; Jiang, K.; Hsu, G. C.; Gao, H.; Lee, K. L.; Yang, A. C.; Yu, X.; Simon, D. I.; Steinmetz, N. F. Shaping Bio-Inspired Nanotechnologies to Target Thrombosis for Dual Optical-Magnetic Resonance Imaging. J. Mater. Chem. B 2015, 3 (29), 6037-6045. https://doi.org/10.1039/C5TB00879D.
(37) Wen, A. M.; Shukla, S.; Saxena, P.; Aljabali, A. A. A.; Yildiz, I.; Dey, S.; Mealy, J. E.; Yang, A. C.; Evans, D. J.; Lomonossoff, G. P.; Steinmetz, N. F. Interior Engineering of a Viral Nanoparticle and Its Tumor Homing Properties. Biomacromolecules 2012, 13 (12), 3990-4001. https://doi.org/10.1021/bm301278f.
(38) Ortega-Rivera, O. A.; Shin, M. D.; Chen, A.; Beiss, V.; Moreno-Gonzalez, M. A.; Lopez-Ramirez, M. A.; Reynoso, M.; Wang, H.; Hurst, B. L.; Wang, J.; et al., Trivalent Subunit Vaccine Candidates for COVID-19 and Their Delivery Devices. J. Am. Chem. Soc. 2021, 143 (36), 14748-14765. https://doi.org/10.1021/jacs.1c06600.
(39) Bruckman, M. A.; Steinmetz, N. F. Chemical Modification of the Inner and Outer Surfaces of Tobacco Mosaic Virus (TMV). Methods Mol Biol 2014, 1108, 173-185. https://doi.org/10.1007/978-1-62703-751-8_13.
(40) Liu, D.; Ding, Z.; Wu, M.; Xu, W.; Qian, M.; Du, Q.; Zhang, L.; Cui, Y.; Zheng, J.; Chang, H.; et al., The Apolipoprotein A-I Mimetic Peptide, D-4F, Alleviates Ox-LDL-Induced Oxidative Stress and Promotes Endothelial Repair through the ENOS/HO-1 Pathway. Journal of Molecular and Cellular Cardiology 2017, 105, 77-88. https://doi.org/10.1016/j.yjmcc.2017.01.017.
(41) He, D.; Zhao, M.; Wu, C.; Zhang, W.; Niu, C.; Yu, B.; Jin, J.; Ji, L.; Willard, B.; Mathew, A. V.; et al., Apolipoprotein A-1 Mimetic Peptide 4F Promotes Endothelial Repairing and Compromises Reendothelialization Impaired by Oxidized HDL through SR-B1. Redox Biol 2018, 15, 228-242. https://doi.org/10.1016/j.redox.2017.11.027.
(42) Rosenbaum, M. A.; Chaudhuri, P.; Abelson, B.; Cross, B. N.; Graham, L. M. Apolipoprotein A-I Mimetic Peptide Reverses Impaired Arterial Healing after Injury by Reducing Oxidative Stress. Atherosclerosis 2015, 241 (2), 709-715. https://doi.org/10.1016/j.atherosclerosis.2015.06.018.
(43) Bastaki, K. M.; Tarlton, J. M. R.; Lightbody, R. J.; Graham, A.; Martin, P. E. Homo Sapiens (Hsa)-MicroRNA (MiR)-6727-5p Contributes to the Impact of High-Density Lipoproteins on Fibroblast Wound Healing In Vitro. Membranes (Basel) 2022, 12 (2), 154. https://doi.org/10.3390/membranes12020154.

1. A multivalent nanoparticle-peptide conjugate, comprising one or more of a biomimetic ApoAI peptide chemically conjugated to an outer surface of a nanoparticle.

2. The conjugate of claim 1, wherein the nanoparticle is selected from a virus-based nanoparticle, a protein nanoparticle, or a nanoparticle that is not spherical and has an aspect ratio of greater than 1.

3. The conjugate of claim 2, wherein the precursor or virus-based nanoparticle is selected from a spherical, virus-like particle (VLP) or a rod-shaped virus nanoparticle (VNP).

4. The conjugate of claim 1, wherein the virus-based nanoparticle is selected from a nanoparticle from a virus selected from a tobacco mosaic virus, a virus from the Virgaviridae family, a virus from the family Potyviridae, a virus from the family Potato virus X, a virus from the Pepino mosaic virus, a virus from the family Alphaflexiviridae, or a mutant of each thereof.

5. The conjugate of claim 1, wherein the nanoparticle comprises a precursor nanoparticle chemically conjugated to a plurality of polyethylene glycol (PEG) molecules.

6. The conjugate of claim 1, wherein the ApoAI peptide is selected from: 4FN (SEQ ID NO: 1), 4FC (SEQ ID NO: 2), or 23MJ (SEQ ID NO: 3), or an equivalent of each thereof.

7. The nanoparticle of claim 5, wherein the PEG molecules are selected from: SM(PEG)₂, SM(PEG)₄, SM(PEG)₆, or SM(PEG)₈.

8. The conjugate of claim 1, wherein the one or more biomimetic ApoAI peptides are chemically conjugated to the outer surface of the nanoparticle by a maleimide-thiol reaction.

9. The conjugate of claim 1, wherein the nanoparticle is selected from a bacteriophage Qβ (Qβ) nanoparticle or a mutant thereof, a tobacco mosaic virus (TMV) nanoparticle or a mutant thereof, or a TMV-Lys (TMV) nanoparticle.

10. The conjugate of claim 9, wherein the nanoparticle is a QB nanoparticle or a mutant thereof and the ApoAI peptide is selected from 4FN (SEQ ID NO: 1), 4FC (SEQ ID NO: 2), or 23MJ (SEQ ID NO: 3), or an equivalent of each thereof.

11. The conjugate of claim 9, wherein the nanoparticle is a TMV nanoparticle or a mutant thereof, or a TMV-Lys nanoparticle, and the ApoAI peptide is selected from 4FN (SEQ ID NO: 1), 4FC (SEQ ID NO: 2), or 23MJ (SEQ ID NO: 3), or an equivalent of each thereof.

12. The conjugate of claim 9, further comprising PEG molecules that are selected from SM(PEG)₂, SM(PEG)₄, SM(PEG)₆, or SM(PEG)₈.

13. A plurality of multivalent nanoparticle-peptide conjugates of claim 1, wherein the plurality of a nanoparticle conjugates in the plurality are the same of different from each other.

14. A composition comprising the multivalent nanoparticle-peptide conjugate of claim 1, and a carrier, optionally a pharmaceutically acceptable carrier.

15. A method for one or more of the following:
a) increasing cholesterol efflux in a subject,
b) reducing reactive oxygen species in a subject,
c) increasing endothelial cell healing rate in a subject,
d) ameliorating an atherosclerosis disease state in a subject, or
e) ameliorating a cardiovascular disease state in a subject,
comprising administering the multivalent nanoparticle-peptide conjugate of claim 1, to the subject in need thereof, thereby providing one or more of:
increasing cholesterol efflux in a subject,
reducing reactive oxygen species in a subject,
increasing endothelial cell healing rate in a subject,
ameliorating an atherosclerosis disease state in a subject, or
ameliorating a cardiovascular disease state in a subject.

16-17. (canceled)

18. The method of claim 15, wherein the subject has one or more of the following conditions: atherosclerosis, cardiovascular disease, or unsafe LDL cholesterol levels.

19. A method for treating one or more of atherosclerosis, cardiovascular disease, or unsafe LDL cholesterol levels in a subject in need thereof, comprising administering to the subject in need thereof of an effective amount of the multivalent nanoparticle-peptide conjugate of claim 1, thereby providing one or more of treating one or more of atherosclerosis, cardiovascular disease, or unsafe LDL cholesterol levels.

20-21. (canceled)

22. A kit comprising one or more of the multivalent nanoparticle-peptide conjugate of claim 1, and optionally instructions for use.


SEQUENCE LISTING (12 sequences)

SEQ ID NO: 1 - protein, 23 aa, synthetic construct
CSGGGDWFKAFYDKVAEKFKEAF

SEQ ID NO: 2 - protein, 23 aa, synthetic construct
DWFKAFYDKVAEKFKEAFGGGSC

SEQ ID NO: 3 - protein, 23 aa, synthetic construct
CGVLESFKASFLSALEEWTKKLQ

SEQ ID NO: 4 - protein, 159 aa, Tobacco mosaic virus
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 5 - protein, 158 aa, synthetic construct
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPK

SEQ ID NO: 6 - protein, 133 aa, unidentified
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 7 - protein, 4 aa, synthetic construct
CGGG

SEQ ID NO: 8 - protein, 5 aa, synthetic construct
CSGGG

SEQ ID NO: 9 - protein, 4 aa, synthetic construct
GGGS

SEQ ID NO: 10 - protein, 18 aa, synthetic construct
DWFKAFYDKVAEKFKEAF

SEQ ID NO: 11 - protein, 22 aa, Homo sapiens
PYSDELRQRLAARLEALKENGG

SEQ ID NO: 12 - protein, 22 aa, Homo sapiens
PVLESFKVSFLSALEEYTKKLN